SEQUENCE LISTING (2 sequences)

SEQ ID NO: 1 - DNA, 12243 bp, Artificial
  Green-fluorescent-protein-reporter fusion gene
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccctcgagttaattaattgtgctagctatctcaagcttcttaatccttttatatctcaatttctcctgctttcactcgaaggatctgaggtttcactcacagccaccttcccccaattacctaaatgaagccatgtgtcataaatatgacacccctaaagagagaggtgatacctatcccctctgtattatcaaccagcatcagacccacggggcttctggcatttcctctggagcctttccgggggtggggaggagtgatctgccaaatctctctcctcttttcccaccccctccctcctcatcacccctctgctgcctgtggctgccactggctgagcatccaactggcccaggaccagataatcagattatcctgagtccagaataatctattccatatggtgtgccagctcggtggctccaagagcgagaggtgtgaggagggccagtgggggtctgggtgggaggctcccctgggagccccaggaacctcagagagccttactgggcagacgtgtaggaaggttggtggggaagaggaacaggaagggagaaactgggtcctctgttgctgggccttttaaggtgatgtgccttgcatcgtctgtgatttaaaggggtgaaggaccaaggggaggggagggggtggcctctcttctgtgccctttctgtctctgaattgtggggaggtactttaataattttgaaattttaaaaacatttatccttattgtatatatagggtgtacacgcacacccacacacgtaggctcacacttgcaggtatgtgaagggccgtctgtcaaggacaccttatgggcccttggattaaggatcaaactctagctgcctgtcttggaagcaaccggctttatccagagagctatctcatgagcccagagtgtcatatccggattctctggctccatcttgcagaacttctggccactgtggtcctggctagggcctagaaacctctttctttcgctgtctttttcaaggctctggtatgtgaagtaaggcctgcccaggctgtgccccaagacagagattaagagacaccttctctggactcttctagctgagacatgggaaaacctctcaaggctccatgttttcacctgtgtcccctcatcccaggatgctgtgagcaggagacctgccaaggcccgcacaatgagactgaaagaacatgtgtgcgaccagctgagcccctctcccctccatggtggaggggctgcctatcttactggcacccacagtgtggagcaagacgaggacattaacccacattggccacactctacctgggaggcttaggcttgcccaggagtgatggtgctgagatagcccacgagtctgacactgtaattaactagtgggatctgctgaggctaagcaggctagtggtggaatcggagtagcttccttcttcttctggggcctctggggctcagggattagggactgtgcatagcacctttgattagcaaggctgtgtcagaaggcctggcaggggaaatgagggcactggggctgcagagtgggtgggagtatggtgcccagctccagcatgaaggctgaggttggaccctgagcctgaaagggggcctccctgtcctgtctgccccctgcctggctgaggcttgacctctcacctctgttctgatggggtggctgcaaggcttcacttcccagccttacacattggcaaccaggggaggctcgggccaggggagcagaagccgctggctctggcacttgtcaaaacacaggccccaaaagtgtgaaggccttggcccagcggggaggctgtgtgggccttgcagcctggagaactcattaactccaagcagctcccaaaggaacccactgaagacactggccagataaggagatcaaaggggcagaggaagcaggcccccccccccttgcaccagctggcccgctgaggtagtttcccagaggccacaggggcaacagtggtaacagagggagggactccacgtggcactccagtgtgttagtctaagccctcaagacttctgggagggaattggtggcaggacgtgccatctgcctgtctctcatgcagtccctcaccgactcactaactctgtgccttttcaactttccctcatgtctgacaccaccatcttcccctgcttcgttcacagcctccctctcctgttccagatccttctgtgtttctgacactcaatgctaaccattaggcctcagctctgaggcttcataagtgtcataagtgttgtgccttcaacgaagtgtggcgtcccccacccctccctagtccaaacatgtctgcctaactttgtagttaataaaatacaacttcaaagttctattaacaagatgcaaacaataagcaatgccagttcggtctggagtaagcaggtagaagctgcagaagcaggcttgtcagggcatttgctgtgccctctgaacccagacactttttaattttaaaccatgtacatctactgtgttttcaatttgtgaaagatcacaagatctttcagtgaaatttctcaattttttttttgatatgatgtgaaatcctggccacatgggggtggagggtatttgagacacttgggtgtcctcagagctgagaccttgcccctcctgcctatgggggctaccgtgtacactcccaccctgtctaagcacacaacaggtctcccgctgtttgttcgagcatcaactaagaggcataatccctgtgcttgaactccaggctcttgttcagataatgagccccaagaaggcatccatttgcctcggttcagaccagatagacctcatggacagctgcagcccgagaaggggcctcctgggccaggcacacaagttcacggcatgggctccacttctccactccaccgtggaagtgagtactgcctctctgggtgtccttgtgggcatggtccttctgacagagaggcaaggatggagaagtgtttgaggaggtcacaggcagtagagaacaaagaacaaaggcatggtagtgggcagagatgcccagagaatcaggtaccaatccattgagtctccaccaccagaaaccatcacccatctggaagacattggggtgcaaacattcagggcactgggagatctgagtgccctcctccagaggaagaaagaggttcagatgtaataccagacaccaggaacctggtggcctagctggagaagagacagcaggtgtgatgtccgcactaggtcaagggtatgaagcaatgcccaaggagttctcccatccttcgtctgctgtacgccaagcgagtgctccatcagccctattattattttgagacggggagtcactaagttatccatgctggccttgaacttactcggtagcccaggacgttgtggctctcctgcctcagctcctcaaacagctgatcccagatctgttctgtcaggctcaagggtttgtttttgagatgtgcctgaaactcactctgcagttgagaatattttttcaattttctgtgtgtactagggctttgcctccatatatgtttgtgctctgtatgcttgtgtagtacatagaggccagaagagtacaatccctagaaatgtcgttatagacagctatgagccaccgtgtggatgctgggaatcagtcaaacctgggtctcgaggaagagcggccagtacttccttcgtttgtttgtttgttttgttattgttgtttgtttgtttgttttcaagacagggtttctctgtatagccctggttgtcctgaaactcacctctagactaggctggcctcaaactcatagatctgcctgcctctgcctccccagtgctgggatcaaaggtgtgctccaacatacctagctgagtagccagtgttcttaactactgagccataactctaggccagaggtgatcttgaacttgaacttctggtcctcctatcttcatctctccagtgctggggttacaagccagtgctacacgtggttatgcagtgttggaataagacctagggcttccggcaggctaggcaagcactctaccgactgaaccacaccaccagccctctaataggtgagacagtccagtagcataggatggcttcaaattcactttgcagccaagactggcccaaactcctcttgccttcattttccaaatgttggaattacaggcatgtgcgtgtatgcccaacttctggagactttattaataagcatgtcacatataagtggggacaggtttgctttaatctctcaatttgtatttatttctctttatttttccaacctagtcccttcattcagtacaacactgaacaggtatggtaagagcaggcatcctcaacagccaggctcacaactgcagtaagctgaggcagcaggatcaggagctaaaggccagctgggttagagtaagtttgaggctagcctgggctacatgagaccatgtttcaagatgtgggaggaagaacactggagagacggctcagcagttaagaagagcacatgttgctcttgaagaacccaaggttctgttcccagaagccacctcaggcagcttacaattgcctgcaaggcccgatcctaaggaacgtgacacctctggcctgtgtgggggcaccagcacacacacacacacacacacacacacacacacacacacacacacatctttaaaaaataaaaggcagcctatgaacctaacaggtagcatcctatgaaatagattataaaactgccattgggacagatgaaggtttctgcttttcagggtttttgggttttgttttgttttgttttgttttttccaacacaggatcttatgtagcccatgttaacctgggccatatacagctaggcctggccttgaactcctgaccctcctgccttcacctcccaagtgtgccaccagctcaaggagttgagttttgtcaaaggactttttactcactgtagctgaccaaagcagcagagctccagtcatgggagggctgggaaggggaccaagctccccatcttgccaagactagaagtgacttgtagtgtggggaggcaggctcacctctccccctgcccggacaccccagcaacctcacaggtcggtcgggatggtttcttcaatgtgcctcccatagcctggacatcagggaggactacaggccacatccaagcctctgggaaggactgaactagagttctctccgtgtctagaggtccctagtccacagttggccaatgcaatcccgaagtctcttgttatacaatttgtaaattaaagtgatggaaaaaaatggcgtagggtccctaataaatgaaggtgtaccctaagcctaggagctagacttcagttctctgagcatggttctttcaactggcagtcctggccataggtgcctgacttactgaggccccaaatatggtccaggtggagcagactggactaaggggtcctgcacctcagagcccctgctatggttcaaagggttcaggccctgcccccctcctggctcgagccggctttgatatgctgatgaattctggaaggggcccgcttcacacctagggctcaggataaagctacagcggacccaatggtcaggcctaagggcgaattctgcagatatcaagcttgtcgacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaagatctgcgactctagaggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgcgactctagaggatcataatcagccataccacatttgtagaggttttacttgctttaaaaaacctcccacacctccccctgaacctgaaacataaaatgaatgccattggttgntgntaacttgtttattggcagcttataatgggtncaaataaagcaatagcatcacaaatttcacaaataaagcatttttttcactgcattctagttgtggttggtccaaactcatcaatgtatcttatcatgtctggatctgngactgtagaggatcataatcagccataccacatttgagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctcgacaaggtacctagagcggccagcttgggctgcaatcgaattccgaagttcctattctctagaaagtataggaacttcaggtctgaagaggagtttacgtccagccaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttcgaagttcctattctctagaaagtataggaacttcatcagtcaggtacataatataacttcgtataatgtatgctatacgaagttattaggtggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - DNA, 12365 bp, Artificial
  Luciferase reporter fusion gene
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccctcgagttaattaattgtgctagctatctcaagcttcttaatccttttatatctcaatttctcctgctttcactcgaaggatctgaggtttcactcacagccaccttcccccaattacctaaatgaagccatgtgtcataaatatgacacccctaaagagagaggtgatacctatcccctctgtattatcaaccagcatcagacccacggggcttctggcatttcctctggagcctttccgggggtggggaggagtgatctgccaaatctctctcctcttttcccaccccctccctcctcatcacccctctgctgcctgtggctgccactggctgagcatccaactggcccaggaccagataatcagattatcctgagtccagaataatctattccatatggtgtgccagctcggtggctccaagagcgagaggtgtgaggagggccagtgggggtctgggtgggaggctcccctgggagccccaggaacctcagagagccttactgggcagacgtgtaggaaggttggtggggaagaggaacaggaagggagaaactgggtcctctgttgctgggccttttaaggtgatgtgccttgcatcgtctgtgatttaaaggggtgaaggaccaaggggaggggagggggtggcctctcttctgtgccctttctgtctctgaattgtggggaggtactttaataattttgaaattttaaaaacatttatccttattgtatatatagggtgtacacgcacacccacacacgtaggctcacacttgcaggtatgtgaagggccgtctgtcaaggacaccttatgggcccttggattaaggatcaaactctagctgcctgtcttggaagcaaccggctttatccagagagctatctcatgagcccagagtgtcatatccggattctctggctccatcttgcagaacttctggccactgtggtcctggctagggcctagaaacctctttctttcgctgtctttttcaaggctctggtatgtgaagtaaggcctgcccaggctgtgccccaagacagagattaagagacaccttctctggactcttctagctgagacatgggaaaacctctcaaggctccatgttttcacctgtgtcccctcatcccaggatgctgtgagcaggagacctgccaaggcccgcacaatgagactgaaagaacatgtgtgcgaccagctgagcccctctcccctccatggtggaggggctgcctatcttactggcacccacagtgtggagcaagacgaggacattaacccacattggccacactctacctgggaggcttaggcttgcccaggagtgatggtgctgagatagcccacgagtctgacactgtaattaactagtgggatctgctgaggctaagcaggctagtggtggaatcggagtagcttccttcttcttctggggcctctggggctcagggattagggactgtgcatagcacctttgattagcaaggctgtgtcagaaggcctggcaggggaaatgagggcactggggctgcagagtgggtgggagtatggtgcccagctccagcatgaaggctgaggttggaccctgagcctgaaagggggcctccctgtcctgtctgccccctgcctggctgaggcttgacctctcacctctgttctgatggggtggctgcaaggcttcacttcccagccttacacattggcaaccaggggaggctcgggccaggggagcagaagccgctggctctggcacttgtcaaaacacaggccccaaaagtgtgaaggccttggcccagcggggaggctgtgtgggccttgcagcctggagaactcattaactccaagcagctcccaaaggaacccactgaagacactggccagataaggagatcaaaggggcagaggaagcaggcccccccccccttgcaccagctggcccgctgaggtagtttcccagaggccacaggggcaacagtggtaacagagggagggactccacgtggcactccagtgtgttagtctaagccctcaagacttctgggagggaattggtggcaggacgtgccatctgcctgtctctcatgcagtccctcaccgactcactaactctgtgccttttcaactttccctcatgtctgacaccaccatcttcccctgcttcgttcacagcctccctctcctgttccagatccttctgtgtttctgacactcaatgctaaccattaggcctcagctctgaggcttcataagtgtcataagtgttgtgccttcaacgaagtgtggcgtcccccacccctccctagtccaaacatgtctgcctaactttgtagttaataaaatacaacttcaaagttctattaacaagatgcaaacaataagcaatgccagttcggtctggagtaagcaggtagaagctgcagaagcaggcttgtcagggcatttgctgtgccctctgaacccagacactttttaattttaaaccatgtacatctactgtgttttcaatttgtgaaagatcacaagatctttcagtgaaatttctcaattttttttttgatatgatgtgaaatcctggccacatgggggtggagggtatttgagacacttgggtgtcctcagagctgagaccttgcccctcctgcctatgggggctaccgtgtacactcccaccctgtctaagcacacaacaggtctcccgctgtttgttcgagcatcaactaagaggcataatccctgtgcttgaactccaggctcttgttcagataatgagccccaagaaggcatccatttgcctcggttcagaccagatagacctcatggacagctgcagcccgagaaggggcctcctgggccaggcacacaagttcacggcatgggctccacttctccactccaccgtggaagtgagtactgcctctctgggtgtccttgtgggcatggtccttctgacagagaggcaaggatggagaagtgtttgaggaggtcacaggcagtagagaacaaagaacaaaggcatggtagtgggcagagatgcccagagaatcaggtaccaatccattgagtctccaccaccagaaaccatcacccatctggaagacattggggtgcaaacattcagggcactgggagatctgagtgccctcctccagaggaagaaagaggttcagatgtaataccagacaccaggaacctggtggcctagctggagaagagacagcaggtgtgatgtccgcactaggtcaagggtatgaagcaatgcccaaggagttctcccatccttcgtctgctgtacgccaagcgagtgctccatcagccctattattattttgagacggggagtcactaagttatccatgctggccttgaacttactcggtagcccaggacgttgtggctctcctgcctcagctcctcaaacagctgatcccagatctgttctgtcaggctcaagggtttgtttttgagatgtgcctgaaactcactctgcagttgagaatattttttcaattttctgtgtgtactagggctttgcctccatatatgtttgtgctctgtatgcttgtgtagtacatagaggccagaagagtacaatccctagaaatgtcgttatagacagctatgagccaccgtgtggatgctgggaatcagtcaaacctgggtctcgaggaagagcggccagtacttccttcgtttgtttgtttgttttgttattgttgtttgtttgtttgttttcaagacagggtttctctgtatagccctggttgtcctgaaactcacctctagactaggctggcctcaaactcatagatctgcctgcctctgcctccccagtgctgggatcaaaggtgtgctccaacatacctagctgagtagccagtgttcttaactactgagccataactctaggccagaggtgatcttgaacttgaacttctggtcctcctatcttcatctctccagtgctggggttacaagccagtgctacacgtggttatgcagtgttggaataagacctagggcttccggcaggctaggcaagcactctaccgactgaaccacaccaccagccctctaataggtgagacagtccagtagcataggatggcttcaaattcactttgcagccaagactggcccaaactcctcttgccttcattttccaaatgttggaattacaggcatgtgcgtgtatgcccaacttctggagactttattaataagcatgtcacatataagtggggacaggtttgctttaatctctcaatttgtatttatttctctttatttttccaacctagtcccttcattcagtacaacactgaacaggtatggtaagagcaggcatcctcaacagccaggctcacaactgcagtaagctgaggcagcaggatcaggagctaaaggccagctgggttagagtaagtttgaggctagcctgggctacatgagaccatgtttcaagatgtgggaggaagaacactggagagacggctcagcagttaagaagagcacatgttgctcttgaagaacccaaggttctgttcccagaagccacctcaggcagcttacaattgcctgcaaggcccgatcctaaggaacgtgacacctctggcctgtgtgggggcaccagcacacacacacacacacacacacacacacacacacacacacacacatctttaaaaaataaaaggcagcctatgaacctaacaggtagcatcctatgaaatagattataaaactgccattgggacagatgaaggtttctgcttttcagggtttttgggttttgttttgttttgttttgttttttccaacacaggatcttatgtagcccatgttaacctgggccatatacagctaggcctggccttgaactcctgaccctcctgccttcacctcccaagtgtgccaccagctcaaggagttgagttttgtcaaaggactttttactcactgtagctgaccaaagcagcagagctccagtcatgggagggctgggaaggggaccaagctccccatcttgccaagactagaagtgacttgtagtgtggggaggcaggctcacctctccccctgcccggacaccccagcaacctcacaggtcggtcgggatggtttcttcaatgtgcctcccatagcctggacatcagggaggactacaggccacatccaagcctctgggaaggactgaactagagttctctccgtgtctagaggtccctagtccacagttggccaatgcaatcccgaagtctcttgttatacaatttgtaaattaaagtgatggaaaaaaatggcgtagggtccctaataaatgaaggtgtaccctaagcctaggagctagacttcagttctctgagcatggttctttcaactggcagtcctggccataggtgcctgacttactgaggccccaaatatggtccaggtggagcagactggactaaggggtcctgcacctcagagcccctgctatggttcaaagggttcaggccctgcccccctcctggctcgagccggctttgatatgctgatgaattctggaaggggcccgcttcacacctagggctcaggataaagctacagcggacccaatggtcaggcctaagggcgaattctgcagatatcaagcttgtcgacatggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaagagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagaattccgaagttcctattctctagaaagtataggaacttcaggtctgaagaggagtttacgtccagccaagctagcttggctgcaggtcgtcgaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcctgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttcgaagttcctattctctagaaagtataggaacttcatcagtcaggtacataatataacttcgtataatgtatgctatacgaagttattaggtggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

(19) United States
(12) Patent Application Publication
(10) Pub. No.: US 2012/0301445 A1
(43) Pub. Date: Nov. 29, 2012

(54) TOOLS FOR ISOLATING AND FOLLOWING CARDIOVASCULAR PROGENITOR CELLS

(75) Inventors: Cedric Blanpain, Lasne (BE); Antoine Bondue, Bruxelles (BE)

(73) Assignee: Universite Libre de Bruxelles, Bruxelles (BE)

(21) Appl. No.: 13/574,218

(22) PCT Filed: Dec. 31, 2010
(86) PCT No.: PCT/EP2010/070959

§ 371 Date: Jul. 19, 2012

(30) Foreign Application Priority Data
Jan. 26, 2010 (EP) 10151610.2
Jan. 26, 2010 (EP) 10151611.0

(51) Int. Cl. A61K 35/34; C12N 5/077; C12N 5/0775; A61P 9/06; C12N 5/074; G01N 21/64; A61P 9/00; A61P 9/10; C12N 5/071; C12N 5/0735

(52) U.S. Cl. 424/93.7; 435/377; 435/366; 435/7.21; 435/6.1; 435/325

(57) ABSTRACT

The present invention provides new methods and tools for isolating pluripotent cardiovascular progenitors (MCPs), by transiently detecting the cell-surface expression of genes upregulated by Mesp1. Cells obtained by the method and there uses in research and clinical settings are also part of the invention. Using genome wide transcriptional analysis, the inventors found upstream and downstream members of the Mesp1 signaling pathway, which form potential new targets for both therapy and for the identification of MCPs and differentiation of MCPs into cardiovascular cells. This invention provides novel and important insights into the molecular mechanisms of cardiovascular specification and provides potential novel methods for dramatically increasing the number of cardiovascular cells for cellular therapy in humans.

### FIELD OF THE INVENTION

The present invention relates to tools and methods for isolating multipotent cardiovascular progenitor cells (MCPs) as well as the use of said MCPs or the differentiated cells arising from the differentiation of MCPs for therapeutic and research purposes.

### BACKGROUND OF THE INVENTION

During cardiovascular development, multipotent cardiovascular progenitors (MCPs) are generated soon after gastrulation and will give rise upon differentiation to all types of cells that constitute a mature heart (Kattman et al., 2006 Dev Cell 11, 723-32; Yang et al., 2008, Nature; Moretti et al., 2006, Cell 127, 1151-65 (2006); Bu et al., 2009, Nature 460, 113-7; Wu et al., 2006, Cell 127, 1137-50). These progenitors have been initially characterized based on Nkx2.5 or Isl1 expression for the progenitors of the first and second heart field respectively (Moretti et al., 2006, Cell 127, 1151-65; Bu et al., 2009, Nature 460, 113-7; Wu et al., 2006, Cell 127, 1137-50), or based on a combination of Flk1 (VEGFR2) and Brachyury expression for early mesodermal progenitors (Kattman et al., 2006 Dev Cell 11, 723-32; Yang et al., 2008, Nature 460, 113-7). Although the existence of MCPs is now well documented, the existence of a common progenitor for primary and second heart fields remains unclear and a better knowledge of the molecular mechanisms leading to their specification represents an important biological question with tremendous clinical and industrial applications. Indeed, the identification of a molecular signature and cell surface markers associated with their specification opens new avenues for the isolation of MCPs during stem cell differentiation, and for the generation of cell preparations with a high cardiovascular potential.

Mesp1 is a b-HLH transcription factor rapidly and transiently expressed in early mesodermal cells that exit the primitive streak to migrate to cardiac forming regions and required for normal cardiac development (Saga et al., 1999, Development 126, 3437-47; Kitajima et al., 2000, Development 127, 3215-26). Mesp1 expression is considered as the first sign of cardiovascular development (Saga et al., 2000, Trends Cardiovasc Med 10, 345-52). Using embryonic stem cells (ES cells) differentiation as a model of cardiovascular development, we recently showed that transient expression of Mesp1 in ES cells can dramatically accelerate and enhance cardiovascular differentiation. We also demonstrated that Mesp1 functions in this process by controlling directly the expression of all known cardiovascular transcription factors and thus acts as a master regulator of MCP specification and/or differentiation (Bondue, et al., 2008, Cell Stem Cell 3, 69-84; David et al., 2008, Nat Cell Biol 10, 338-45; Lindsley et al., 2008, Cell Stem Cell 3, 55-68; Wu 2008, Cell Stem Cell 3, 1-2).

The need for cardiovascular progenitors or adult cardiovascular cells is very high in both clinical and research settings and is currently not easily fulfilled. The knowledge that Mesp-1 is a key regulator in cardiovascular differentiation of ES cells is indeed very important, but not sufficient in providing effective tools of isolating suitable progenitor cells without e.g. genetic manipulation enabling the visualization of Mesp-1 expressing ES cells. The present invention overcomes this problem by unraveling the signaling pathways and cellular markers associated with cardiovascular differentiation of stem cells, preceding or following said Mesp-1 expression in ES cells.

The current invention thus provides improved tools and methods to obtain cardiovascular progenitors in a simplified manner, based on certain surface markers that coincide or preclude Mesp-1 expression leading to the MCP phenotype. Said isolated MCP cells can then be further differentiated into any type of cardiovascular cells which can be safely transplanted in patients with cardiovascular diseases or used in research and industrial perspectives. The tools and methods according to the invention are of special interest for isolating human MCPs, because no genetic manipulation of ES cells is needed in order to isolate the MCPs.

### SUMMARY OF THE INVENTION

As a starting point, the inventors isolated the earliest MCPs that are generated during ES cells differentiation to define the cellular and molecular properties that are associated with the early steps of MCP specification. To this end, recombinant ESC lines were generated that expressed a reporter gene (e.g. green fluorescent protein or Luc.) under control of the regulatory region of Mesp1. It was subsequently seen that Mesp1 expressing cells are highly enriched for MCPs that can give rise to different types of cardiac cells (atrial, ventricular, conduction cells), endothelial cells and smooth muscle cells.

In a next step, these early MCPs were transcriptionally profiled and a series of genes that are preferentially expressed in MCPs, and that can be used to isolate MCP from various sources were identified. These gene profiles will enable us to specifically enhance cardiovascular cells production and to influence the types of cardiovascular cells generated from ES cells.

The present invention thus provides a method for isolating multipotent cardiovascular progenitors (MCPs) from a group of stem cells comprising:

a) culturing of mammalian stem cells in a medium containing suitable agents allowing their proliferation and maintaining their pluripotenty,
b) differentiating the mammalian stem cells obtained in step a) towards cardiovascular progenitors cells, and
c) isolating those cells of step b) that express the following markers: Flk1, PDGFRa, and CXCR4,
wherein said stem cells are preferably embryonic stem cells (ES cells), preferably human embryonic stem cells, pluripotent stem cells, haematopoietic stem cells, totipotent stem cells, mesenchymal stem cells, induced pluripotent stem cells (iPS) or adult stem cells, adult heart, epicardial, vessel or muscular cells.

Preferably, said isolation step is performed by means of cell-sorting using labeled binding molecules, such as fluorescently labeled, magnetically labeled or density labeled binding molecules, such as binding molecules selected from the group of: specific antibodies, aptamers, small molecules, peptides, carbohydrates, nucleic acids, peptide-nucleic acids, or small organic molecules.

In a preferred embodiment of the method of the present invention, said selection of MCPs is done at day 3 of stem cells differentiation. Most preferably, said isolated MCPs are capable of differentiating into both primary and secondary heart field cells.

The invention further provides a kit for isolating, visualising or identifying MCPs at day 3 of stem cell differentiation, wherein said MCPs are capable differentiating into both primary and secondary heart field cells, comprising:

a) binding molecule(s) specific for the Flk1 marker on the cell surface of a cell,
b) binding molecule(s) specific for the PDGFRa marker on the cell surface of a cell, and
c) binding molecule(s) specific for the CXCR4 marker on the cell surface of a cell.

In addition, the invention provides for the use of the kit according to the invention, for isolating, visualising or identifying MCPs at day 3 of stem cell differentiation, wherein said MCPs are capable differentiating into both primary and secondary heart field cells, wherein said kit comprises:

a) binding molecule(s) specific for the Flk1 marker on the cell surface of a cell,
b) binding molecule(s) specific for the PDGFRa marker on the cell surface of a cell, and
c) binding molecule(s) specific for the CXCR4 marker on the cell surface of a cell.

In the kit according to the present invention, claim the binding molecules are preferably selected from specific antibodies, aptamers, small molecules, peptides, carbohydrates, nucleic acids, peptide-nucleic acids, small organic molecules. More preferably, said binding molecules are detectably labeled, preferably fluorescently labeled, magnetically labeled or density labeled.

The invention also provides a substantially purified population of MCPs obtained by the method according to the invention, expressing the following markers on their cell surface: Flk1, PDGFRa, and CXCR4, and capable of differentiating into both primary and secondary heart field cells. The invention additionally provides a composition comprising the substantially pure population of human cardiovascular precursor cells according to the invention.

Alternatively, the invention provides a method of generating cardiovascular cells such as cardiomyocytes, endothelial cells, and vascular smooth muscle cells comprising the steps of:

a) culturing MCPs obtained according to the method according to the present invention, and
b) allowing said MCP cells to differentiate due to the endogenous expression of Mesp-1. The invention also provides a composition comprising a population of cardiovascular cells produced by said method.

The invention also provides for a method of cardiovascular cell replacement comprising administering to a subject in need of such replacement a composition comprising a population of MCPs according to the present invention, or cardiovascular cells according to the present invention.

The invention thus also provides for a method of treating a disorder characterized by insufficient cardiac function comprising administering to a subject in need of such treatment a composition comprising a population of MCPs or cardiovascular cells provided in the present invention.

The invention further provides for a method for performing cellular therapy, comprising the steps of:

a) providing MCPs or cardiovascular cells provided in the present invention, and
b) injecting said cells into the heart or the vasculature of the subject in need thereof allowing exogenous or autologous cell therapy. Preferably, said cardiovascular function is preferably disturbed due a disease or disorder selected from the group consisting of: Congenital Heart Disease, such as malformations and misplacements of cardiac structures, acquired heart and vascular diseases, such as myocardial infarction, cardiac hypertrophy and cardiac arrhythmia and cardiovascular damage due to trauma.

The “multipotent cardiovascular cells” or “MCPs” isolated and provided by the present invention encompass cardiovascular progenitor cells that are capable of forming essentially all types of cardiovascular cells after differentiation, i.e. both cells of the primary and secondary heart fields.

Preferably, said MCP cells are at a very early stage of development, i.e. Day 3 or 4 of embryonic stem cell differentiation, most preferably of Day 3 of stem cell differentiation.

In a particularly preferred embodiment the expression level of the gene Mesp1 is additionally analysed in order to identify and/or isolate early cardiovascular progenitors.

Another object of the invention is the provision of a new Mesp1-reporter gene construct which was used to transform the ES cells as used above. The invention thus provides a multipotent cardiovascular progenitor reporter gene-construct (called “MCP reporter construct” hereinafter) and its use for detecting MCPs, monitoring the development and differentiation of MCP cells and cardiovascular cells derived therefrom.

Said construct can be used to follow the development and differentiation of MCPs and to better characterize the cellular and molecular mechanisms regulating MCP specification.

In a preferred embodiment, the MCP reporter construct according to the invention comprises a reporter gene such as a GFP or Luc. Gene, cloned under the regulatory region of the Mesp-1 gene. The MCP reporter construct can additionally comprise a selection marker such as an antibiotics resistance gene, known in the art.

In a preferred embodiment, the transformed ES cell line expresses a Venus-GFP reporter under the control of the 5.6 kB upstream of the Mesp1 coding sequence (accession number NM_{—}008588), taking the translation start as a reference (FIG. 1A). This sequence faithfully recapitulates Mesp1 expression in transgenic embryos in vivo (Haraguchi et al Dev 2001). The DNA sequence of the complete Mesp1-VenusGFP plasmid construct used is represented by SEQ ID NO. 1. The linear fragment between Pacl and BamH1 restriction sites was electroporated in E14Tg2a mouse ES cells to generate the Mesp1-GFP cell line. This “Mesp1-GFP” cell line was deposited on Dec. 23, 2010 with the Belgian Co-ordinated Collections of Micro-organisms (BCCM/LMBP) under the provisional deposit number LMBP 8051 CB.

Said selection gene is in preferred embodiments flanked by a recombination site, which enables the excision of the selection gene from the construct. Exemplary site-directed recombination sites are known in the art, e.g. cre-loxP, FRT-FLP, lambda integrase, etc. The inventors here used the Flippase Recognition Target (FRT) sequence, which can be cleaved by the Flippase enzyme. The antibiotic resistance gene can be placed under control of a prokaryotic and/or eukaryotic promoter sequence. The present invention used the pgk/em7 combination of a prokaryotic (em7) and eukaryotic (pgk) promoter to trigger the expression of the neomycin resistance gene.

In a preferred embodiment, the MCP reporter construct is defined by SEQ ID NO. 1 and comprises the Mesp-1 gene regulatory sequence, wherein the GFP-coding sequence is cloned. This part is followed by a neomycin resistance gene, driven by a pgk/em7 promoter duo, flanked by two FRT sites (cf. FIG. 1A and SEQ ID NO.1).

The coding sequence of Mesp-1 is in fact exchanged by the coding sequence of the GFP reporter gene. This has as a result that the endogenous Mesp-1 expression itself is not deregulated at all in the transformed ES cells. Due to the use of the Mesp1 regulatory sequence, the MCP reporter construct follows the Mesp-1 expression pattern and enables visualization of said profile (and thus also the MCP cells expressing it) in real time, without affecting the function of Mesp-1.

The MCP reporter construct of the invention can thus be used for the visualization and detection of MCPs.

In addition, the MCP reporter construct of the invention can be used for real-time imaging of the differentiating or developing MCPs.

The latter aspect is interesting for screening or testing agents or drugs for e.g. their toxic or pharmacological effect on the development of and propagation of MCPs.

In addition, the MCP reporter construct can be used for screening factors or agents that stimulate differentiation of MCPs.

The inventors further generated a Mesp1-Luc reporter to screen with a higher throughput the molecules controlling Mesp1 expression. The construct is constructed similarly to the construct using the Venus/GFP reporter gene, wherein the coding sequence for GFP is replaced by the Luc. coding sequence. Its use is similar to that of the GFP construct but its improved detection capacities result in an easier use for high throughput screening.

The invention further provides a method or an assay for identifying an extrinsic factor (proteins, peptides or small molecules) that promotes MCP-differentiation comprising the steps of:

a) allowing cells to differentiate into MCPs according to the method of the invention, in the presence or absence of said extrinsic factor, and
b) analysing the effect of the extrinsic factor on MCP-differentiation by comparing the number of MCP cells formed in the presence and absence of said extrinsic factor, based on the expression of markers Flk1, PDGFRa, and CXCR4 in said cells (or of Mesp1-reporter gene expression).

The present invention further provides a method for identifying an agent or extrinsic factor that influences MCP-formation or differentiation comprising the steps of:

a) providing genetically modified ES-cells expressing a reporter gene under control of the Mesp-1 regulatory region according to the invention,
b) allowing said ES-cells to differentiate into MCPs according to the method of the invention, in the presence or absence of said agent or extrinsic factor, and
b) analysing the effect of the agent or extrinsic factor on MCP-differentiation by comparing the behaviour of the MCP cells formed in the presence and absence of said agent or extrinsic factor, based on the amount of MCP reporter construct expressed.

The invention further provides an assay for determining the pharmacological properties and the toxicity of any chemical compound or pharmacological agent based on the production of cells obtained by the method of the present invention.

The invention further provides an assay for identifying an extrinsic factor (proteins, peptides or small molecules) that promotes MCP-differentiation comprising the steps of:

a) allowing stem cells to differentiate in vitro, in the presence or absence of said extrinsic factor, and
b) analysing the effect of the extrinsic factor on MCP-differentiation by comparing the number of MCP cells formed in the presence and absence of said extrinsic factor, wherein the number of MCP cells is detected according to the method of the invention.

The invention further provides a method for specifying and/or differentiating Mesp1 expressing MCPs into a particular subset of cardiovascular lineages such as cardiomyocytes, vascular or endothelial cells, by inducing the expression of one or more genes selected from the group comprising Flk1, PDGFRa and CXCR4.

The invention further provides a method for modulating the expression or activity of one or more genes selected from the group comprising Flk1, PDGFRa and CXCR4 to enhance the production and/or the differentiation of stem cells or MCPs towards cardiovascular cell lineages of both ventricular and auricular subtypes, or a method to modulate the differentiation of stem cells specifically to ventricular or auricular subtypes by modulating expression of said one or more genes.

The invention further provides the use of a reporter system based on Mesp1 expression to track and/or quantify MCPs at the time of their specification.

The invention further provides a method of targeting endogenous cardiovascular progenitors in a subject in need thereof, comprising the step of modulating the expression of one or more genes selected from the group comprising Flk1, PDGFRa and CXCR4. In a preferred embodiment, the method is used to specifically target cardiovascular progenitors or specifically target cardiovascular cells in order to restore cardiac function. Preferably, said cardiovascular function is disturbed due a disease or disorder selected from the group consisting of: Congenital Heart Disease, such as malformations and misplacements of cardiac structures, acquired heart and vascular diseases, such as myocardial infarction, cardiac hypertrophy and cardiac arrhythmia and cardiovascular damage due to trauma.

The invention further provides a composition comprising the substantially pure population of human cardiovascular precursor cells obtained by the methods of the invention.

The invention further provides a composition comprising a population of differentiated cardiovascular cells produced by the methods of the invention.

### DESCRIPTION OF THE DRAWINGS

FIG. 1. Engineering ESCs expressing Venus-GFP under the regulatory region of Mesp1.

(A) Schematic representation of the Mesp1 reporter transgene. (B) Detection of GFP in whole mounted EBs in Mesp1-GFP ESCs at D3 of differentiation (right panel). Unmodified ESCs at the same day of differentiation are used as control (left panel). Scale bars, 50 μm. (C and D) Kinetics of Mesp1 mRNA expression measured by RT-qPCR (C), and Mesp1-GFP expression as detected by FACS (D) during ESC differentiation. Results are normalized for Mesp1 expression in undifferentiated ESCs (C) or represent the percentage of Mesp1-GFP positive cells (D). (E) Relative expression of Mesp1 and GFP transcripts in Mesp1-GFP-expressing cells (black bars), and in Mesp1-GFP-non-expressing cells (white bars) isolated by FACS at D3. Results are normalized for the expression of the different transcripts in all sorted cells (grey bars).

FIG. 2. Isolation and functional characterization of early Mesp1-GFP-expressing cells.

(A-C) Expression of cardiovascular markers after 8 days of differentiation of the indicated cell populations isolated at D3 of ESC differentiation. Cardiac and endothelial differentiation were quantified by FACS using a cardiac specific isoform of the TroponinT (cTNT) (A) and the endothelial marker CD31 (B). SMC differentiation was assessed by counting the percentage of cells expressing smooth muscle actin (SMA) on cytospin-slides (C). (D) Relative mRNA expression of cardiovascular markers in Mesp1-GFP positive derived cells (black bars) assessed by real time RT-PCR 8 days after replating. Results are normalized to the expression of the different transcripts in the Mesp1-GFP negative derived cells (white bars), and expression in all sorted cells is shown in grey. (E) Immunostainings for cTNT (CMs), VE-cadherin (ECs) and SMA (SMCs) in individual colonies obtained following the replating at clonal density of isolated Mesp1-GFP cells at D3 and cultured for 13 days. Scale bars, 50 μm. (F) Quantification of colonies expressing cardiovascular (cTNT and VE-cadherin), cardiac (cTNT) and endothelial (VE-cadherin) markers as obtained in (E). (G) RT-PCR analysis of cardiovascular markers in colonies derived from a single Mesp1-GFP isolated cell in 96 wells after 13 days of differentiation. (H) Cardiovascular potential of Mesp1-GFP isolated cells at D3 of ESC differentiation and transplanted under the kidney capsule of NOD/SCID mice. Immunostainings of the graft were performed 4 weeks after transplantation. Cardiovascular differentiation was assessed by immunostainings for cTNT (CMs), VE-cadherin (ECs) and SMA (SMCs).

FIG. 3. Isl1 is expressed in a subset of early Mesp1-expressing cells.

(A, B) Quantification of Mesp1-GFP (A) and Isl1 (B) expression as measured by immunostaining of Mesp1-GFP cells on cytospin-slides at D3 and D4 of ESC differentiation. (C, D) Confocal microscopy analysis of GFP (Mesp1) and Isl1 immunostainings in Mesp1-GFP embryoid bodies at D3 (C) and D4 (D) of ESC differentiation. Right panels represent magnification of the squares and arrows indicate cells that co-express Mesp1 and Isl1 Scale bars, 30 μm. (E, F) Quantification of Isl1 expression in Mesp1-GFP-expressing cells (E), and of GFP (Mesp1) expression in Isl1-expressing cells (F), as measured by immunostaining of Mesp1-GFP cells on cytospin-slides at D3 and D4 of ESC differentiation.

FIG. 4. Isolation and functional characterization of early MCPs using a combination of monoclonal antibodies

(A) Cell surface marker expression in Mesp1-GFP-expressing cells. Real time RT-PCR analysis of the expression of cell surface markers is performed in isolated Mesp1-GFP-expressing cells at D3 of ESC differentiation. Results are normalized for the mRNAs expression in GFP negative cells. (B) Detection of CXCR4, PDGFRa, and Flk1 by FACS at D3, in all living cells (upper panel) and in the Mesp1-GFP population (lower panel). Mesp1-GFP cells express high level of CXCR4, PDGFRa and Flk1. (C) FACS quantification of Mesp1-GFP expression in different cell fractions regarding CXCR4, PDGFRa and Flk1 expression. (D) Multicolor FACS analysis of CXCR4, PDGFRa and Flk1 expression in Mesp1-GFP cells at D3 and D4 of ESC differentiation gated on Mesp1-GFP cells. Percentages of Mesp1-GFP cells in each quadrant are shown and percentages of CXCR4/PDGFRa/Flk1 TP cells are shown in parenthesis. The density plots show that at D3, the majority of Mesp1-GFP cells express high level of CXCR4, PDGFRa and Flk1. (E) Temporal expression of CXCR4, PDGFRa and Flk1 during ESC differentiation as detected by FACS. (F) Enrichment of Mesp1 expression in CXCR4, PDGFRa and Flk1 TP cells at D3, as measured by RT-PCR on FACS isolated cells. Results are normalized for the relative transcript expression in all sorted cells. (G to I) Differentiation potential of CXCR4, PDGFRa and Flk1 TP cells. Cells were isolated at D3 of ESC differentiation and cultured for 8 days. Cardiac and endothelial differentiation were quantified by FACS using antibodies recognizing the cardiac TroponinT (cTNT) (G), and the endothelial marker CD31 (H). SMC differentiation was assessed by counting the percentage of cells expressing smooth muscle actin (SMA) on cytospin-slides (I).

FIG. 5: Cardiovascular and EMT transcription factors in early MCPs

(A, B) Real time RT-PCR analysis of mRNA relative expression of cardiovascular (A) and EMT (B) transcription factors in FACS isolated Mesp1-GFP cells at D3 of ESC differentiation (black bars). Results are normalized for the relative expression of the different transcripts in Mesp1-GFP negative cells (white bars). (C) E-Cadherin expression in all cells and in Mesp1-expressing cells as measured by FACS analysis. Note the strong decrease in E-Cadherin expression in most Mesp1-expressing cells. (D) Real time RT-PCR analysis of the expression of cardiovascular transcription factors in CXCR4/PDGFRa/Flk1 TP cells isolated at D3 (white bars) and D4 (black bars) of ESC differentiation. Results are normalized for the mRNA expression in CXCR4-/PDGFRa-/Flk1-cells.

FIG. 6: Signaling pathways that are required for MCP specification.

(A and B) FACS quantification of Mesp1 expression as measured with the GFP reporter cell line at D3 of differentiation in the presence of serum and inhibitors of Wnt (Dkk1), BMP (Noggin) and Nodal (SB431542) signalling (A), or in serum free conditions in the presence of activators of BMP and Wnt pathways (B). Results are normalized for Mesp1 expression in basal conditions.

FIG. 7. Cooperation of Mesp1 and Hey2 in MCP specification and cardiovascular differentiation

(A) Schematic representation of Mesp1, Hey2 and Mesp1/Hey2 Dox inducible ESCs. (B) FACS plots and quantification of CXCR4, PDGFRa and Flk1 expression in Mesp1, Hey2 and Mesp1/Hey2 expressing cells at D4, 48 hours post Dox addition since D2. Percentages of living cells in each quadrant are shown and percentages of CXCR4+/PDGFRa+/Flk1+ triple positive cells are shown in parenthesis. (C) Quantification of CXCR4, PDGFRa and Flk1 triple positive cells at 24 hours (D3—white bars) and 48 hours (D4—black bars) post Dox addition in Mesp1, Hey2 and Mesp1/Hey2 differentiating ESCs. Mesp1 and Hey2 cooperate to promote the Flk1, PDGFRa and CXCR4 triple positive cells. (D) FACS quantification of cardiac TroponinT expression at D8 of differentiation in Mesp1, Hey2 or Mesp1/Hey2 ESCs, in the presence or not of Dox at D2-D4. Mesp1 and Hey2 act synergistically to promote cardiac differentiation. Data represent the mean and s.e.m. of at least 4 biologically independent experiments. (E) Immunostaining of cardiac Troponin T at D8 of differentiation in Dox inducible Mesp1, Hey2 and Mesp1/Hey2 ESCs in the presence or not of Dox from D2 to D4. Shown images are representative acquisitions of 4 biologically independent experiments, and scale bars represent 500 μm. (F) FACS quantification of CD31 expression at D7 of ESC differentiation in Mesp1, Hey2 and Mesp1/Hey2 Dox inducible ESCs, in the presence or not of Dox from D2 to D4. (G) Immunostainings for VE-Cadherin expression at D7 of differentiation in Dox inducible Mesp1, Hey2 and Mesp1/Hey2 ESCs in the presence or not of Dox from D2 to D4. Scale bar is 100 μm. (H) Real time PCR analysis of the expression of cardiovascular markers at D8 in Mesp1 (white bars), Hey2 (grey bars), and Mesp1/Hey2 Dox inducible ESCs (black bars). Results are normalized for relative expression of transcripts in Dox unstimulated cells. (I) Real time PCR analysis of the expression of cardiovascular transcription factors in inducible Mesp1 (white bars), Hey2 (grey bars), and Mesp1/Hey2 (black bars) differentiating ESCs at D4, 48 hours post Dox addition. Results are normalized for relative expression of the different transcripts in Dox unstimulated cells. Co-expression of Mesp1 and Hey2 lead to a stronger increase in Hand2, Nk×2-5, Tbx20, FoxF1a and Twist1 than Mesp1 alone.

FIG. 8. Hey2 expression and EMT induction in Dox inducible Hey2 ESCs.

(A) Immunostainings of Hey2 expression in Dox inducible Hey2 ESCs in the presence (right panel), or absence (left panel) of Dox for 24 hours. Scale bar is 50 μm. (B) Real time PCR analysis of the expression of EMT markers in Dox inducible Mesp1 (white bars), Hey2 (grey bars), and Mesp1/Hey2 (black bars) ESCs at D4 of differentiation, 48 hours post Dox addition. Results are normalized for relative expression of the different transcripts in Dox unstimulated cells. (C) FACS analysis of E-Cadherin expression in Mesp1, Hey2 and Mesp1/Hey2 Dox inducible cells at D4 of differentiation, 48 h following Dox addition. Mesp1/Hey2 co-expression leads to a stronger decrease in E-cadherin than Mesp1 expression alone, indicating that Mesp1 and Hey2 cooperate to promote EMT during ESC differentiation.

### DETAILED DESCRIPTION OF THE PREFERRED EMBODIMENTS

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

All documents cited in the present specification are hereby incorporated by reference in their entirety. In particular, the teachings of all documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. When specific terms are defined in connection with a particular aspect or embodiment, such connotation is meant to apply throughout this specification, i.e., also in the context of other aspects or embodiments, unless otherwise defined.

The terms “progenitor” or “precursor” refer generally to an unspecialised or relatively less specialised and proliferation-competent cell which can under appropriate conditions give rise to at least one relatively more specialised cell type, such as inter alia to relatively more specialised progenitor cells or eventually to terminally differentiated cells, i.e., fully specialised cells that may be post-mitotic.

As used herein, the term “pluripotent” denotes a stem cell capable of giving rise to cell types originating from all three germ layers of an organism, i.e., mesoderm, endoderm, and ectoderm, and potentially capable of giving rise to any and all cell types of an organism, although not able of growing into the whole organism.

A progenitor or stem cell is said to “give rise” to another, relatively more specialised cell when, for example, the progenitor or stem cell differentiates to become said other cell without previously undergoing cell division, or if said other cell is produced after one or more rounds of cell division and/or differentiation of the progenitor or stem cell.

The terms “ES cell” and “stem cell” are used interchangeably herein and generally refer to pluripotent stem cells of mammalian origin. The term “mammal” refers to any animal classified as such, including, but not limited to, humans, domestic and farm animals, zoo animals, sport animals, pet animals, companion animals and experimental animals, such as, for example, mice, rats, hamsters, rabbits, dogs, cats, guinea pigs, cattle, cows, sheep, horses, pigs and primates, e.g., monkeys and apes.

The term “ES cells” encompasses all kinds of mammalian pluripotent stem cells, including human embryonic stem cells obtained from embryos or from established embryonic stem cell lines which are commercially available or differentiated from human cells (e.g. obtained form the patient) by genetic manipulation as outlined herein. The term “stem cell” is preferably a human stem cell and is undifferentiated prior to culturing and is capable of undergoing differentiation. The stem cell may be selected from the group including, but not limited to, isolated embryonic stem (ES) cells e.g. human embryonic stem cells (hES), established embryonic stem cell lines (e.g. human), pluripotent stem cells, haematopoietic stem cells, totipotent stem cells, mesenchymal stem cells, neural stem cells, or adult stem cells. The stem cell is preferably a human embryonic stem (hES) cell which may be derived directly from an embryo or from a culture of embryonic stem cells. For example, the stem cell may be derived from a cell culture, such as human embryonic stem cells (hES) cells as disclosed in Reubinoff et al., (Nature Biotech. 16:399-404 2000). The stem cells may be derived from an embryonic cell line or embryonic tissue. The embryonic stem cells may be cells which have been cultured and maintained in an undifferentiated state. Such cells have been described in WO2000/027995, WO2001/042421, WO2001/098463 and WO2001/068815, the contents of which are incorporated herein by reference.

Preferred stem cells are pluripotent stem cells derived from any kind of mammalian embryonic tissue, e.g., embryonic, foetal or pre-foetal tissue, the cells being capable under appropriate conditions of producing progeny of different cell types that are derivatives of all three germinal layers, i.e., endoderm, mesoderm, and ectoderm. Included in the definition of stem cells are thus embryonic stem cells of various types, exemplified without limitation by murine embryonic stem cells, e.g., as described by Evans & Kaufman 1981 (Nature 292: 154-6) and Martin 1981 (PNAS 78: 7634-8); rat pluripotent stem cells, e.g., as described by Iannaccone et al. 1994 (Dev Biol 163: 288-292); hamster embryonic stem cells, e.g., as described by Doetschman et al. 1988 (Dev Biol 127: 224-227); rabbit embryonic stem cells, e.g., as described by Graves et al. 1993 (Mol Reprod Dev 36: 424-433); porcine pluripotent stem cells, e.g., as described by Notarianni et al. 1991 (J Reprod Fertil Suppl 43: 255-60) and Wheeler 1994 (Reprod Fertil Dev 6: 563-8); sheep embryonic stem cells, e.g., as described by Notarianni et al. 1991 (supra); bovine embryonic stem cells, e.g., as described by Roach et al. 2006 (Methods Enzymol 418: 21-37); human embryonic stem (hES) cells, e.g., as described by Thomson et al. 1998 (Science 282: 1145-1147); human embryonic germ (hEG) cells, e.g., as described by Shamblott et al. 1998 (PNAS 95: 13726); embryonic stem cells from other primates such as Rhesus stem cells, e.g., as described by Thomson et al. 1995 (PNAS 92:7844-7848) or marmoset stem cells, e.g., as described by Thomson et al. 1996 (Biol Reprod 55: 254-259).

Other types of stem cells are also included in the term as are any cells of mammalian origin capable of producing progeny that includes derivatives of all three germinal layers, regardless of whether they were derived from embryonic tissue, foetal tissue or other sources. Stem cells are not derived from a malignant source. A cell or cell line is from a “non-malignant source” if it was established from primary tissue that is not cancerous, nor altered with a known oncogene. It may be desirable, but not always necessary, that the stem cells maintain a normal karyotype throughout prolonged culture under appropriate conditions. It may also be desirable, but not always necessary, that the stem cells maintain substantially indefinite self-renewal potential under appropriate in vitro conditions. It is expected that the culture conditions and methods as defined herein will be applicable at least to all stem cell lines from the same sources as those tested and suggest that these culture conditions for improved differentiation are applicable to all stem cell lines and stem cells in general. Furthermore, the fact that these differentiation conditions can be established without fetal calf serum, and thus without the potential presence of animal pathogens, increases the chance that these hES-derived embryonic germ layer derivatives such as ectoderm, mesoderm or endoderm, more preferably cardiomyocytes or cardiac mesoderm are suitable for transplantation in patients preferably with heart disease.

Where administration of MCPs or mature cardiovascular cells to a patient is contemplated, it may be preferable that the stem cells, e.g., human ES or EG cells, subjected to the methods of the present invention, are selected such as to maximise the tissue compatibility between the patient and the administered cells, thereby reducing the chance of rejection of the administered cells by patient's immune system (graft vs. host rejection). For example, advantageously the stem cells or cell lines may be typically selected which have either identical HLA haplotypes (including one or preferably more HLA-A, HLA-B, HLA-C, HLA-D, HLA-DR, HLA-DP and HLA-DQ; preferably one or preferably all HLA-A, HLA-B and HLA-C to the patient, or which have the most HLA antigen alleles common to the patient and none or the least of HLA antigens to which the patient contains pre-existing anti-HLA antibodies.

Alternatively, the stem cells suitable for use in the present methods may be derived from a patient's own tissue. This would enhance compatibility of differentiated tissue grafts derived from the stem cells with the patient. The stem cells may be first genetically modified prior to use through introduction of genes that may control their state of differentiation prior to, during or after their exposure to the factors that contribute to the promotion of cardiovascular differentiation of the stem cells. They may be genetically modified through introduction of vectors expressing factors or using a combination of vectors and chemical agents that induced pluripotent states such as Oct4, Sox2, Nanog, or Klf4, or selectable marker under the control of a stem cell specific promoter such as Oct-4 or of genes that may be upregulated to induce differentiation such as Mesp1. The stem cells may be genetically modified at any stage with markers or gene so that the markers or genes are carried through to any stage of cultivation. The markers may be used to purify the differentiated or undifferentiated stem cell populations at any stage of cultivation.

Preferred “human ES cells” are described by Thomson et al. 1998 (supra) and e.g. in U.S. Pat. No. 6,200,806. The scope of the term covers pluripotent stem cells that are derived from a human embryo at the blastocyst stage, or before substantial differentiation of the cells into the three germ layers. ES cells, in particular hES cells, are typically derived from the inner cell mass of blastocysts or from whole blastocysts. Derivation of hES cell lines from the morula stage has been documented and ES cells so obtained can also be used in the invention (Strelchenko et al. 2004. Reproductive BioMedicine Online 9: 623-629). As noted, prototype “human EG cells” are described by Shamblott et al. 1998. Such cells may be derived, e.g., from gonadal ridges and mesenteries containing primordial germ cells from foetuses. In humans, the foetuses may be typically 5-11 weeks post-fertilisation.

Those skilled in the art will appreciate that, except where explicitly required otherwise, the term stem cells may include primary tissue cells and established lines that bear phenotypic characteristics of the respective cells, and derivatives of such primary cells or cell lines that still have the capacity of producing progeny of each of the three germ layers.

Exemplary but non-limiting established lines of human ES cells include lines which are listed in the NIH Human Embryonic Stem Cell Registry (http://stemcells.nih.gov/research/registry), and sub-lines thereof, such as, lines hESBGN-01, hESBGN-02, hESBGN-03 and hESBGN-04 from Bresagen Inc. (Athens, Ga.), lines Sahlgrenska 1 and Sahlgrenska 2 from Cellartis AB (Göteborg, Sweden), lines HES-1, HES-2, HES-3, HES-4, HES-5 and HES-6 from ES Cell International (Singapore), line Miz-hES1 from MizMedi Hospital (Seoul, Korea), lines I 3, I 3.2, I 3.3, I 4, I 6, I 6.2, J 3 and J 3.2 from Technion—Israel Institute of Technology (Haifa, Israel), lines HSF-1 and HSF-6 from University of California (San Francisco, Calif.), lines H1, H7, H9, H13, H14 of Wisconsin Alumni Research Foundation/WiCell Research Institute (Madison, Wis.), lines CHA-hES-1 and CHA-hES-2 from Cell & Gene Therapy Research Institute/Pochon CHA University College of Medicine (Seoul, Korea), lines H1, H7, H9, H13, H14, H9.1 and H9.2 from Geron Corporation (Menlo Park, Calif.), lines Sahlgrenska 4 to Sahlgrenska 19 from Göteborg University (Göteborg, Sweden), lines MB01, MB02, MB03 from Maria Biotech Co. Ltd. (Seoul, Korea), lines FCNCBS1, FCNCBS2 and FCNCBS3 from National Centre for Biological Sciences (Bangalore, India), and lines RLS ES05, RLS ES 07, RLS ES10, RLS ES13, RLS ES15, RLS ES 20 and RLS ES 21 of Reliance Life Sciences (Mumbai, India). Other exemplary established hES cell lines include those deposited at the UK Stem Cell Bank (http://www.ukstemcellbank.org.uk/), and sub-lines thereof, e.g., line WT3 from King's College London (London, UK) and line hES-NCL1 from University of Newcastle (Newcastle, UK) (Strojkovic et al. 2004. Stem Cells 22: 790-7). Further exemplary ES cell lines include lines FC018, AS034, AS034.1, AS038, SA111, SA121, SA142, SA167, SA181, SA191, SA196, SA203 and SA204, and sub-lines thereof, from Cellartis AB (Göteborg, Sweden).

Further within the term stem cells are cells obtainable by manipulation, such as inter alia genetic and/or growth factor mediated manipulation, of non-pluripotent mammalian cells, such as somatic and particularly adult somatic mammalian cells, including the use of induced pluripotent stem (iPS) cells, as taught inter alia by Yamanaka et al. 2006 (Cell 126: 663-676) and Yamanaka et al. 2007 (Cell 131: 861-872).

A skilled person will appreciate that further cell lines having characteristics of mammalian, esp. mouse or human, pluripotent stem cells (mPS), especially ES cells or EG cells, may be established in the future, and these may too be suitable in the present invention.

A skilled person can also use techniques known in the art to verify that any established or yet to be established mPS cell lines, or sub-lines thereof, show desirable cell characteristics, such as expansion in vitro in undifferentiated state, preferably normal karyotype and ability of pluripotent differentiation.

Stem cells or cell lines or cultures thereof are described as “undifferentiated” when a substantial proportion (for example, at least 60%, preferably at least 70%, even more preferably at least 80%, still more preferably at least 90% and up to 100%) of cells in the stem cell population display characteristics (e.g., morphological features or markers) of undifferentiated stem cells, clearly distinguishing them from cells undergoing differentiation. Undifferentiated stem cells are generally easily recognised by those skilled in the art, and may appear in the two dimensions of a microscopic view with high nuclear/cytoplasmic ratios and prominent nucleoli. It is understood that colonies of undifferentiated cells within the population may often be surrounded by neighbouring cells that are more differentiated. Nevertheless, the undifferentiated colonies persist when the population is cultured or passaged under appropriate conditions known per se, and individual undifferentiated cells constitute a substantial proportion of the cell population. Undifferentiated stem cells may express the stage-specific embryonic antigens (SSEA) 3 and 4, and markers detectable using antibodies designated Tra-1-60 and Tra-1-81 (Thomson et al. 1998, supra). Undifferentiated stem cells may also typically express Oct-4 and TERT.

Within the present specification, the terms “differentiation”, “differentiating” or derivatives thereof denote the process by which an unspecialised or relatively less specialised cell, such as, for example, stem cell or progeny thereof, becomes relatively more specialised. In the context of cell ontogeny, the adjective “differentiated” is a relative term. Hence, a “differentiated cell” is a cell that has progressed further down a certain developmental pathway than the cell it is being compared with. The differentiated cell may, for example, be a terminally differentiated cell, i.e., a fully specialised cell capable of taking up specialised functions in various tissues or organs of an organism, which may but need not be post-mitotic; or the differentiated cell may itself be a progenitor cell within a particular differentiation lineage which can further proliferate and/or differentiate. A relatively more specialised cell may differ from an unspecialised or relatively less specialised cell in one or more demonstrable phenotypic characteristics, such as, for example, the presence, absence or level of expression of particular cellular components or products, e.g., RNA, proteins or other substances, activity of certain biochemical pathways, morphological appearance, proliferation capacity and/or kinetics, differentiation potential and/or response to differentiation signals, electrophysiological behaviour, etc., wherein such characteristics signify the progression of the relatively more specialised cell further along the said developmental pathway. The terms “cardiac differentiation”, “cardiomyogenic differentiation”, “cardiomyogenesis” or “differentiating stem cells into cardiomyocytes” means the formation of cardiomyocytes from stem cells preferably from hES cells. Formation of cardiomyocytes is defined by the formation of contracting EBs, contracting seeded cells, immune cytological staining for cardiomyocyte specific marker, and expression of cardiomyocyte specific marker.

The term “medium permissive to differentiation of stem cells” means that the medium does not contain components, in sufficient quantity, which would suppress stem cell differentiation or would cause maintenance and/or proliferation of stem cells in undifferentiated or substantially undifferentiated state. By means of illustration, such components absent from the medium may include leukaemia inhibitory factor (LIF), basic fibroblast growth factor (b-FGF), and/or embryonic fibroblast feeders or conditioned medium of such feeders, depending on the particular stem cell type.

In certain embodiments, the medium may comprise basal medium formulations generally known in the art. Suitable basal media formulations include, without limitation, Minimum Essential Medium (MEM), alpha modified Minimum Essential Medium (alpha-MEM), Basal Medium Essential (BME), Dulbecco's Modified Eagle's Medium (DMEM), F-12 Nutrient Mixture (Ham; see, e.g., Ham 1965. PNAS 53: 288), Neurobasal medium (NM; see, e.g., Brewer et al. 1993. J Neurosci Res 35: 567-76), and the like, which are commercially available (e.g., Invitrogen, Carlsbad, Calif.). Compositions of basal media such as above are known per se and contain ingredients necessary for mammalian cell development. For example, such ingredients may include inorganic salts (preferably at least salts containing Na, K, Mg, Ca, Cl, P, and possibly Cu, Fe, Se and Zn), physiological buffers (e.g., HEPES, bicarbonate or phosphate buffers), amino acids, vitamins, and sources of carbon (e.g. glucose, or pyruvate, e.g., sodium pyruvate), and may optionally also comprise reducing agents (e.g., glutathione), nucleotides, nucleosides and/or nucleic acid bases, ribose, deoxyribose, etc. In addition, the media may be further supplemented with one or more compounds of interest, including without limitation additional L-glutamine, non-essential amino acids, β-mercaptoethanol, protein factors such as insulin, transferrin or bovine serum albumin, antibiotic and/or antimycotic components, such as, e.g., penicillin, streptomycin and/or amphotericin, or other components.

Culturing the stem cells can be done in the presence of a medium that is substantially free of xeno- and serum-components and thus comprises a clinically compliant medium, free of contaminants such as bacteria, viruses, growth factors, allergens, prions, etc.

In the present work, the knowledge that Mesp1 expression is indicative for cardiovascular cell fate was used. By following the Mesp-1 expression in a genetically modified ES cell-line, the inventors managed to isolate early MCPs (i.e. D3 or 4 of ESC differentiation) at the time of their MCP specification and that are common for both first and second heart fields, and identified the early molecular events associated with said specification.

Based on their isolation followed by expression profiling and functional characterization of their progeny, the inventors could show that Mesp1 expressing cells indeed represent an early cardiovascular progenitor for all cardiovascular lineages.

A molecular signature was subsequently identified composed by the expression of a small subset of cardiovascular transcription factors that is associated with early MCP specification. The inventors further showed that MCPs represent a homogenous population that co-express Flk1, PDGFRa and CXCR4 at the time of their specification, and that these cell surface markers can be used to prospectively isolate MCPs during ESC differentiation without the requirement of genetically modified cells.

The comprehensive analysis of the earliest molecular mechanisms controlling cardiovascular commitment of ES cells and the cell surface markers identified can be used to allow the generation of cell preparations with a high cardiovascular potential for cardiac cell therapy, with ability to give rise to derivatives of all cardiac cells including first and second heart field derivatives, and generate ventricular, auricular, pace maker as well as conductive cell types, but also vascular endothelial and smooth muscle cell fates.

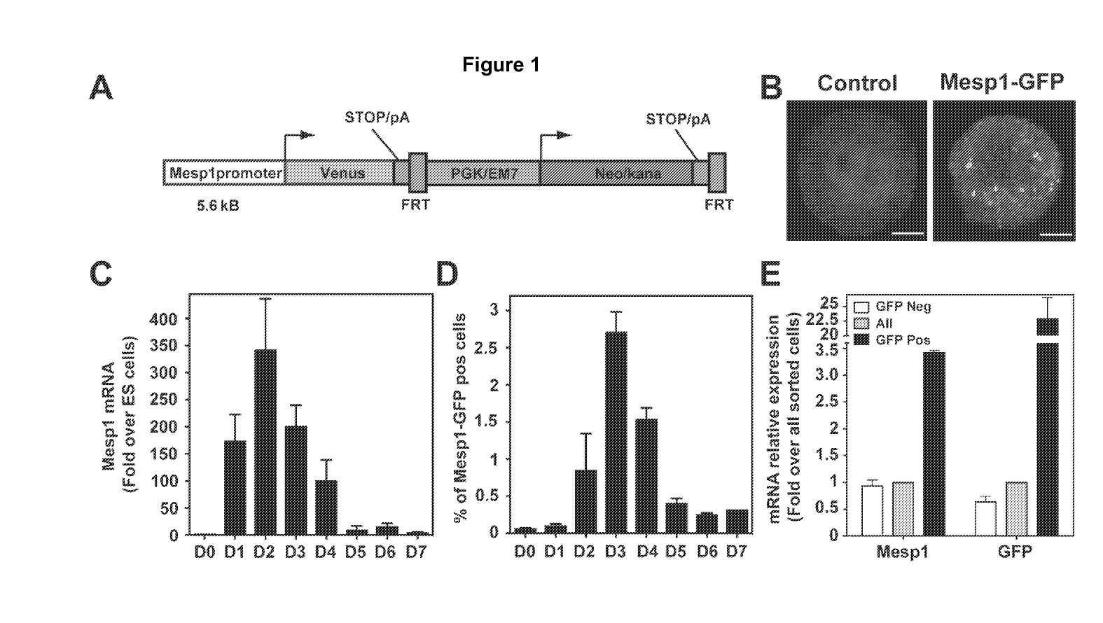

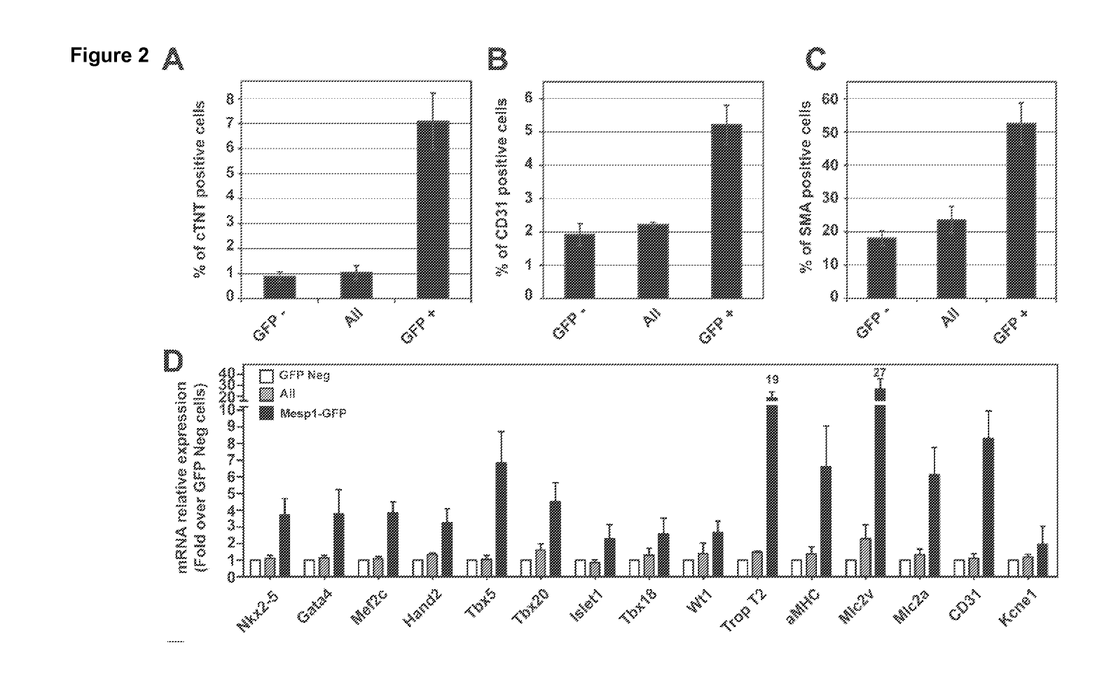

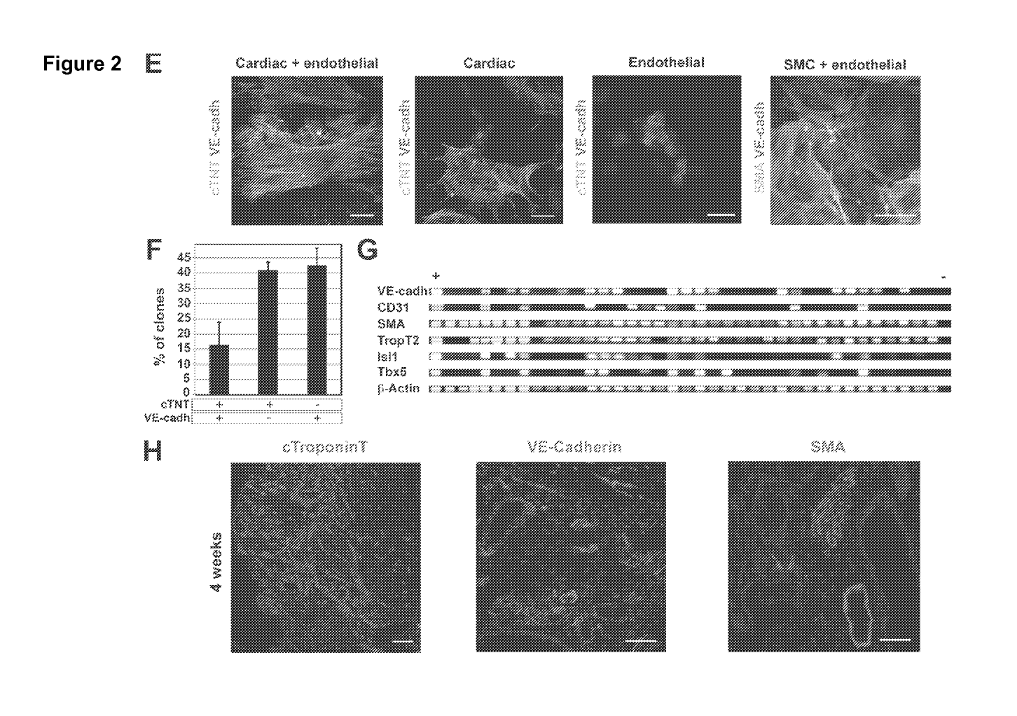

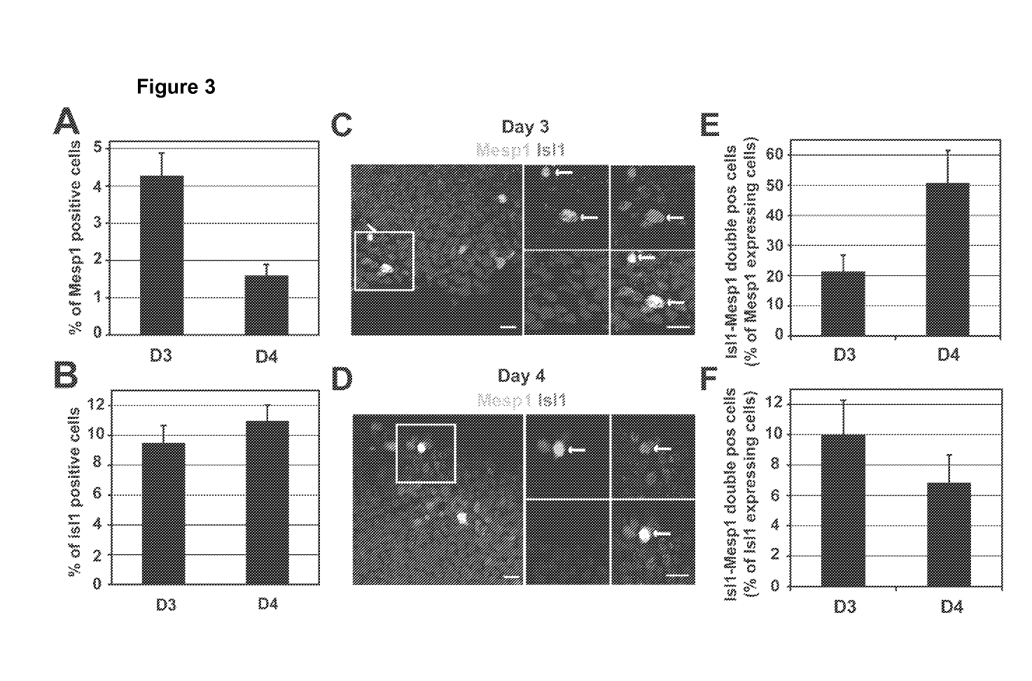

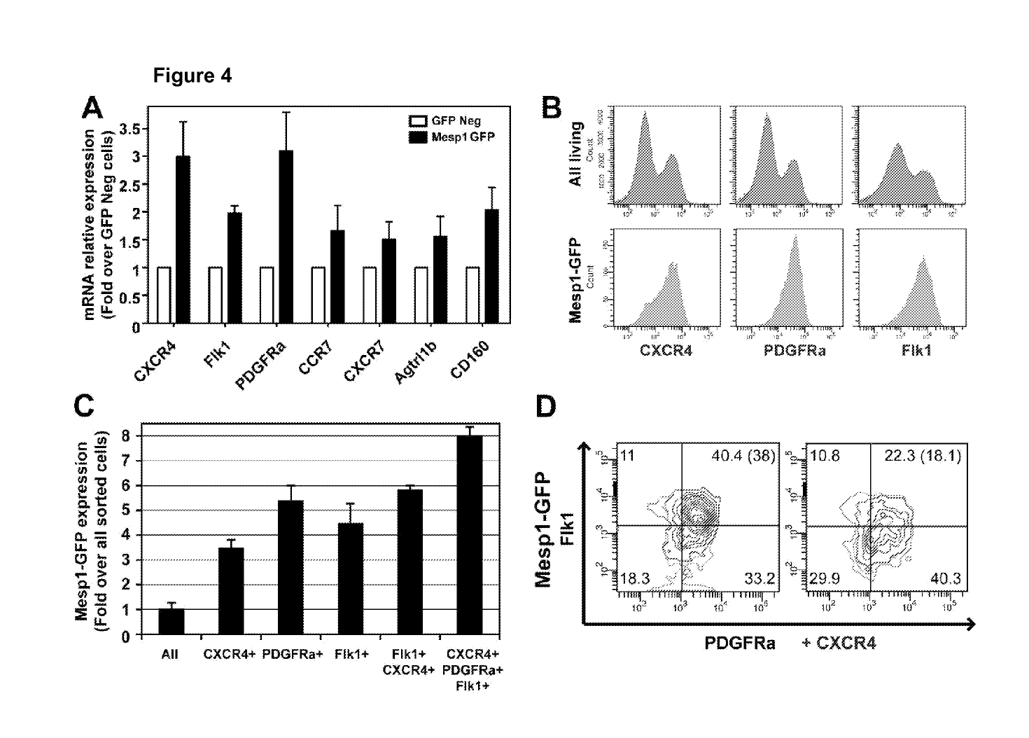

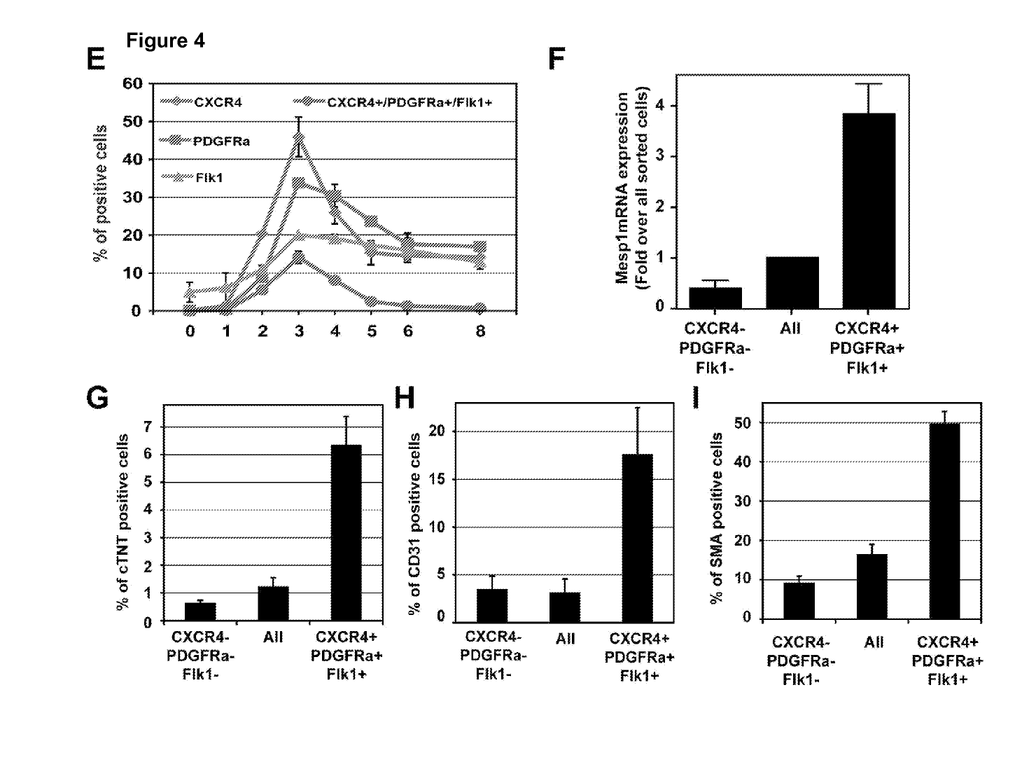

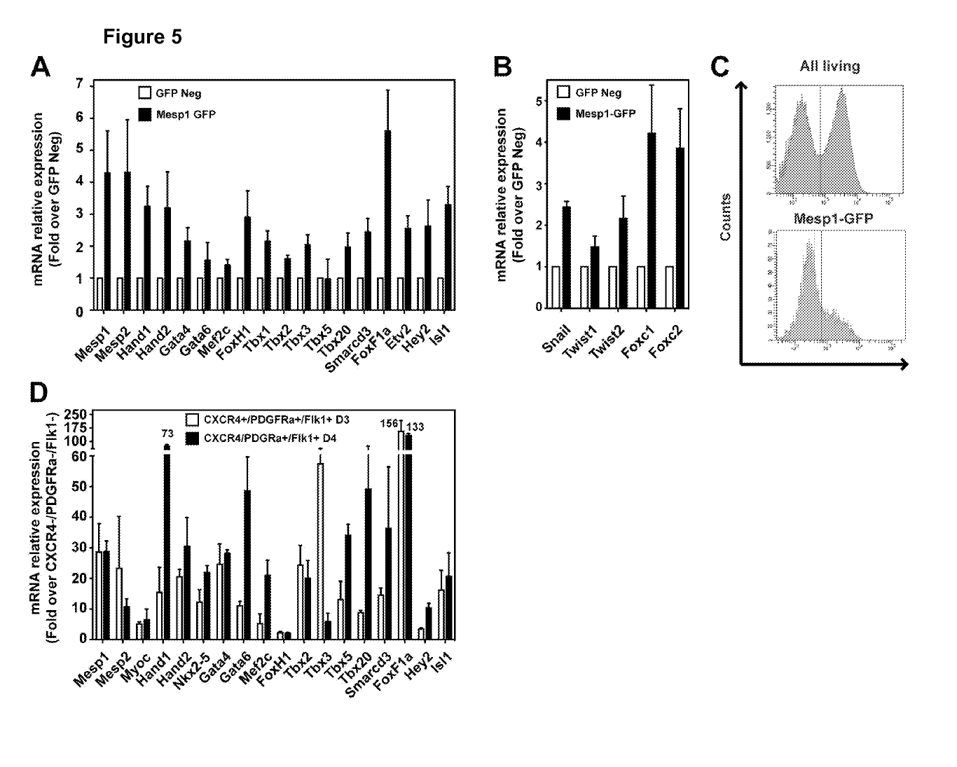

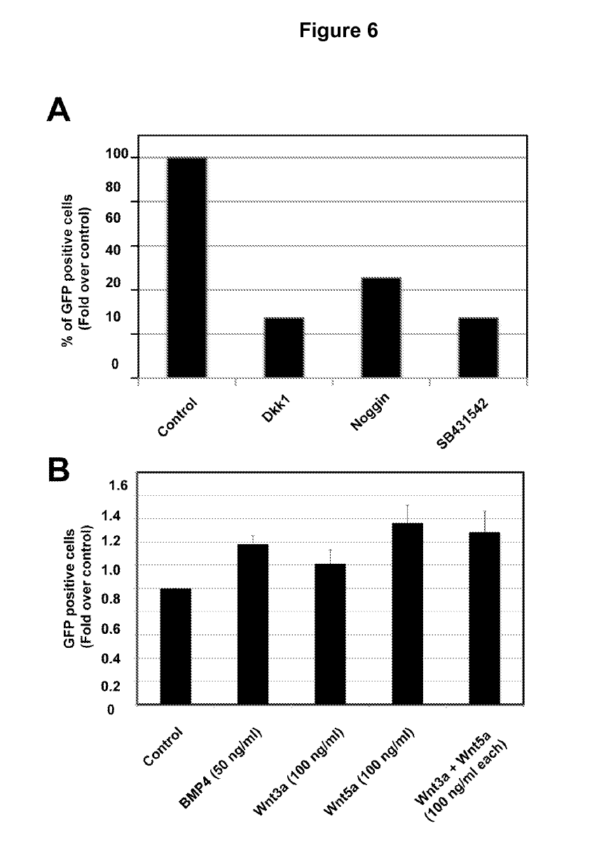

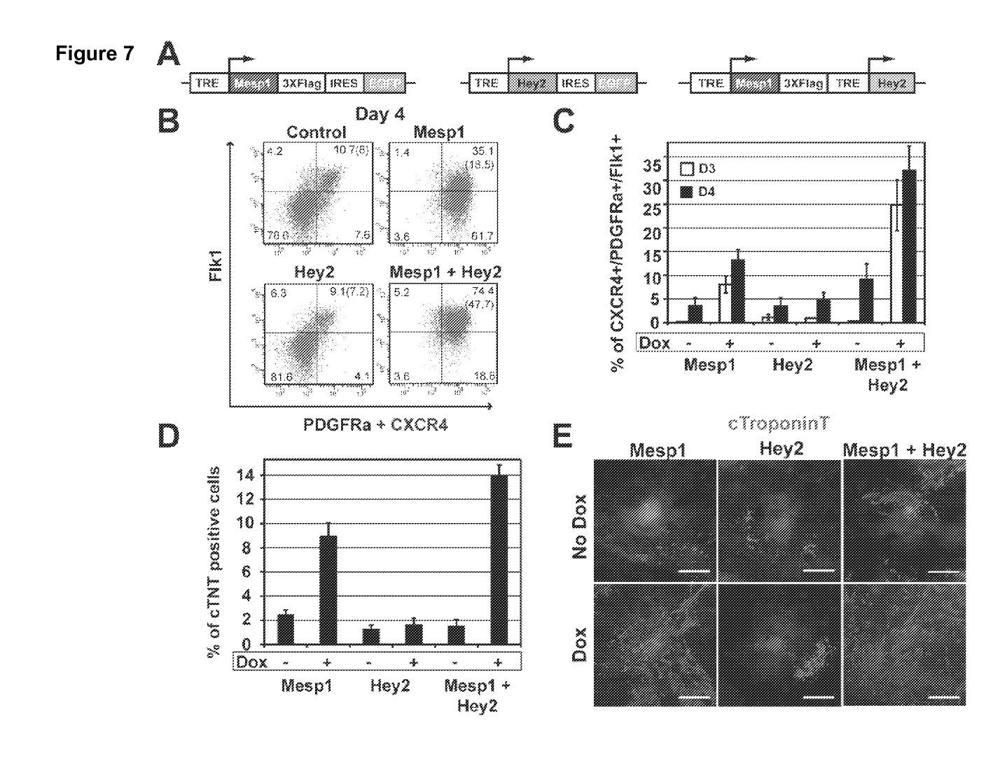

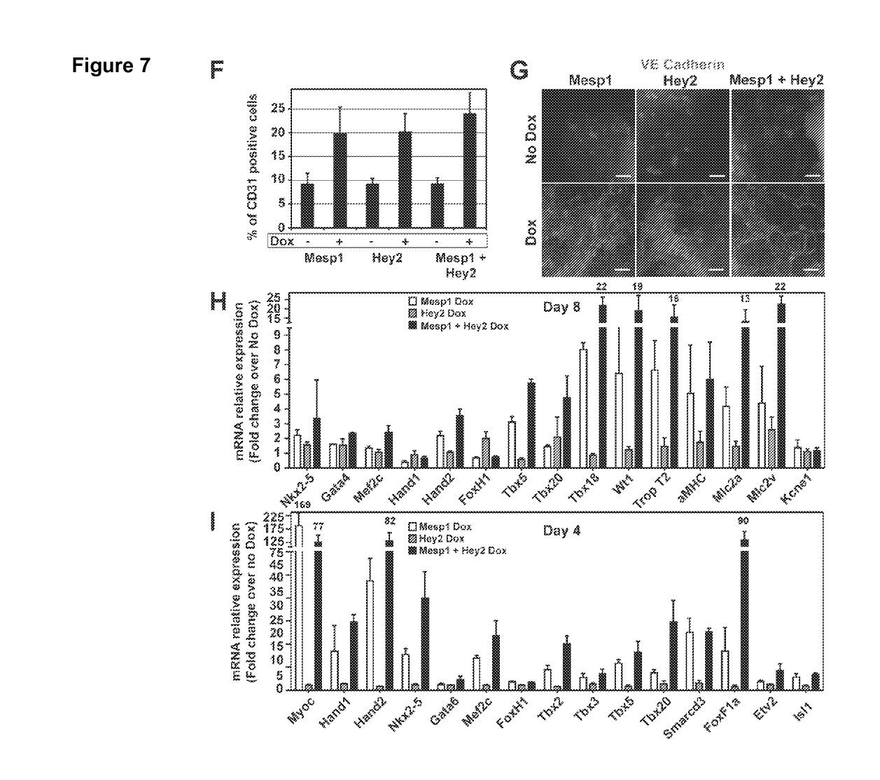

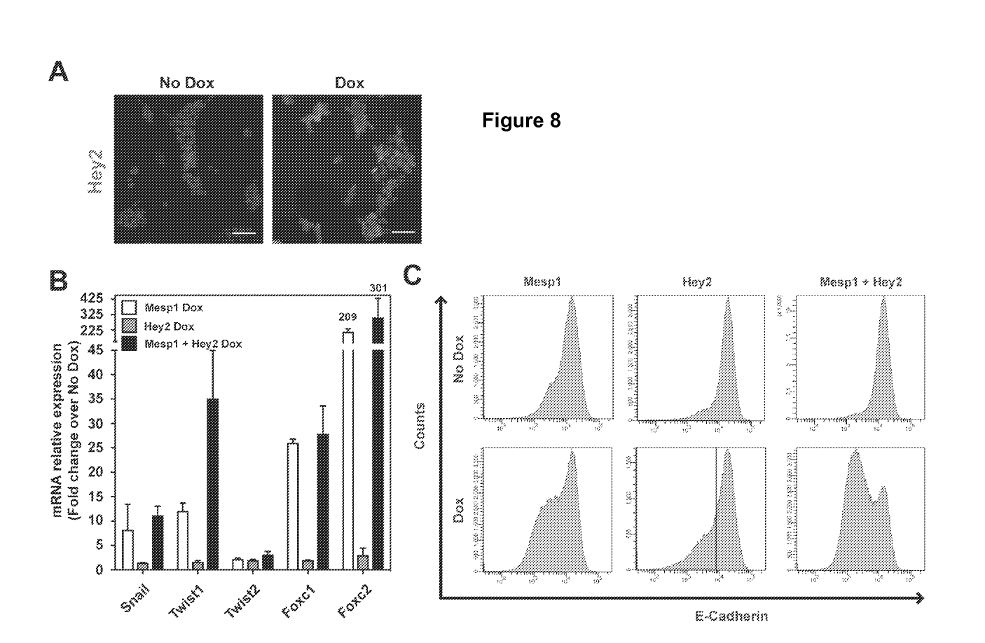

To better characterize the cellular and molecular mechanisms regulating MCP specification, an ESC line that expresses a Venus-GFP reporter under the control of the 5.6 kB upstream of the Mesp1 coding sequence (accession number NM_{—}008588), taking the translation start as a reference (FIG. 1A) is provided by the present invention. This sequence faithfully recapitulates Mesp1 expression in transgenic embryos in vivo (Haraguchi et al Dev 2001). The DNA sequence of the complete Mesp1-VenusGFP plasmid construct used is represented by SEQ ID NO. 1. The linear fragment between Pacl and BamH1 restriction sites was electroporated in E14Tg2a mouse ES cells to generate the Mesp1-GFP cell line. This “Mesp1-GFP” cell line was deposited on Dec. 23, 2010 with the Belgian Co-ordinated Collections of Micro-organisms (BCCM/LMBP) under the provisional deposit number LMBP 8051 CB.

The inventors showed that temporal expression of Mesp1 mRNA, which starts to be expressed at D2, peaks at D3 and is followed by a rapid extinction (FIGS. 1B, 1C and 1D). No GFP positive cell was observed in undifferentiated ESCs. Mesp1-GFP positive cells appeared between D2 and D3 during ESC differentiation, and at D3, 2.6% of differentiating ESCs expressed the GFP transgene (FIG. 1D). After D4, the percentage of GFP positive cells rapidly decreased (FIG. 1D). Using RT-PCR analysis, inventors showed that Mesp1 and GFP transcripts are enriched in Mesp1-GFP-expressing cells isolated at the peak of Mesp1-GFP expression (D3) during ESC differentiation (FIGS. 1, D and E), demonstrating that the Mesp1-GFP reporter ESC line recapitulates the temporal endogenous expression of Mesp1 and can be used to isolate Mesp1-expressing cells.

To evaluate the enrichment in cardiac progenitors, the beating potential of these Mesp1-GFP cells was compared with Mesp1 negative fractions and beating areas were detectable only in the GFP positive fraction, but not in the GFP negative cells, suggesting that the cardiac progenitor cells were located exclusively in the Mesp1-GFP expressing cells. To quantify this cardiac enrichment, the expression of a cardiac isoform of troponinT (cTNT) was measured by FACS and showed that the isolation of Mesp1-GFP cells allowed a 7 fold enrichment in cardiac cells, showing that Mesp1-GFP cells represent cardiac progenitors (FIG. 2A). The Mesp1-GFP cells also displayed a greater potential for endothelial and smooth muscle cell differentiation (FIGS. 2B and 2C). Altogether, the three main lineages arising from the differentiation of MCPs represent about 65% of all cells in Mesp1-GFP isolated cells. By RT-PCR, we also showed that Mesp1 expressing cells can differentiate into the various cell types of the cardiovascular lineage such as atrial cells (MLC2a), ventricular cells (Mlc2v, Tbx5), conduction and pacemaker cells (KcnE1), epicardial cells (Tbx18, Wt1); and endothelial cells (CD31) (FIG. 2D).

To determine whether Mesp1-expressing cells represent common progenitors for both primary and second heart fields, clonal analysis of Mesp1-expressing cells isolated at D3 of ESC differentiation was performed, after which they were replated at low density to identify the different cell fates that can be generated from the differentiation of single Mesp1-GFP expressing cells. First heart field progenitors are bipotent progenitors that can give rise to cardiac and smooth muscle cells after differentiation (Wu et al. Cell 2006), while second heart field progenitors are tripotent progenitors and are able to generate cardiac, endothelial and smooth muscle cell fates following their differentiation (Moretti et al. Cell 2006, Bu et al. Nature 2009). After differentiation of Mesp1-GFP expressing cells isolated at D3, immunostaining of individual colonies was performed, showing that almost all colonies contained SMA positive cells. About 15% of the clones presented both cardiac and vascular cells, 40% only expressed cTNT and 40% only expressed VE-cadherin (FIGS. 2E and 2F). To determine whether derivatives of both the first and second heart fields are present within the tripotent colonies, single Mesp1-expressing cells were replated and RT-PCR was performed after their differentiation. Similarly to the results obtained by immunostaining, the vast majority of the colonies expressed SMA, among them some colonies also expressed EC or CM markers and some colonies expressed markers of all three lineages, and Tbx5 and Isl1 were both expressed in 40% of the tripotent colonies (FIG. 2G), supporting that a fraction of Mesp1-expressing cells represents common progenitors for both heart fields.

Isolation of Mesp1-GFP-expressing cardiovascular progenitors at D3 of ESC differentiation, followed by their transplantation under the kidney capsule of NOD/SCID mice allowed to identify the cardiovascular potential of Mesp1-GFP MCPs in vivo. Four weeks after transplantation of Mesp1-GFP expressing cells, no teratomas were observed, while Mesp1-GFP negative cells grafted under the other kidney capsule of the heterolateral kidney as control generated teratomas. Immunostainings of the grafts demonstrated that Mesp1-GFP cells mainly differentiated into cardiomyocytes, although expression of endothelial and smooth muscle cell markers were also present within the graft (FIG. 2H).

Isl1 expression has been previously used to mark tripotent MCPs at D5 of ESC differentiation that could represent second heart field progenitors (Moretti et al., 2006). The microarray-based transcriptional profiling of Mesp1-GFP cells as performed herein demonstrated that MCPs preferentially expressed is 11 (FIG. 5A). To better characterize the relation between Mesp1 and Isl1 expression, immunostainings for Isl1 and GFP expression were performed on Mesp1-GFP cells at D3 and D4 following ESC differentiation. By immunofluorescence, Mesp1-GFP was expressed in 4% and 1.5% of cells respectively at D3 and D4 (FIG. 3A). Isl1 expression was lower at D3 than at D4 and that in later stages of differentiation, but could already be detected at the edge of EBs in about 10% of ESCs at D3 and D4 (FIG. 3B). At D3 about 20% of Mesp1-expressing cells co-expressed Isl1 (FIGS. 3C and 3E), while at D4 about 50% of Mesp1-expressing cells co-expressed Isl1 (FIGS. 3D and 3E). The Mesp1/Isl1 double positive cells represented 10% and 6% of Isl1-expressing cells at D3 and D4 of ESC differentiation respectively (FIG. 3F). These observations support the notion that Isl1 a marker tripotent cardiovascular progenitors is expressed together with Mesp1 only in a fraction of early Mesp1-expressing cells, showing that Mesp1 expressing cells represent a common pool of cells for both previously described bipotent (first heart field) and tripotent (second heart field) cardiovascular progenitors.

These observations showed that Mesp1-GFP reporter cell line temporally recapitulates endogenous Mesp1 expression and allows the isolation of Mesp1 expressing cells, which correspond to the earliest MCPs, common for all cardiovascular lineages and contributing to first and second heart field derivatives. These results place Mesp1-expressing progenitors isolated at D3 of ESC differentiation on the top of all other previously described cardiovascular progenitors, such as progenitors of the first (Wu et al. Cell 2006, Nelson et al. Stem Cells 2008) and second heart field (Moretti et al. Cell 2006), that were all isolated at later stage of ESC differentiation (D5-D6).

One of the main challenges in the field is the development of a simple and reproducible approach to isolate MCPs using monoclonal antibodies or other binding agent through e.g. flow cytometry. Such an approach would enable a high-throughput cell sorting of MCPs out of a culture of ES cells or EBs, without the need of genetic manipulation of the cells. The microarray-based transcriptional profiling of Mesp1-GFP cells as performed herein demonstrated that MCPs preferentially expressed some 20 different cell surface markers (FIG. 4A). From said list, we could show that PDFGRa, CXCR4 and Flk1 can be used in combination to specifically isolate Mesp1 expressing MCPs, which opens very interesting perspectives for providing a simple and effective tool for isolating MCPs. By multicolor flow cytometric analysis, it was confirmed that Mesp1-GFP cells are enriched for CXCR4, PDGFRa and Flk1 expression (FIG. 4B). Up to now, these markers have been used at later stages of ES cell differentiation (D5-D6) to isolate specialized subpopulations of cardiovascular progenitors such as progenitors of the first heart field progenitors (Flk1/CXCR4-Nelson et al. Stem Cells 2008), and second heart field progenitors (Moretti et al. Cell 2006), To identify how these cell surface markers can be used alone or in combination to isolate an early common cardiovascular progenitor for both heart fields at D3 of ESC differentiation, flow cytometry was used to show that the combined detection of CXCR4/PDGFRa and Flk1 is the most efficient strategy to detect Mesp1-expressing cells at D3 of ESC differentiation, with better enrichment obtained for Mesp1 than with the previously described combined detection of Flk1 and CXCR4 (Nelson et al. Stem Cells 2008) (FIG. 4C). At D3 of ESC differentiation Mesp1-GFP expressing cells represent a relatively homogenous population co-expressing high levels of CXCR4, PDGFRa and Flk1 (FIG. 4D), whereas at later stages and as soon as 24 hours later (D4), Mesp1 expressing cells are more heterogenous with regard of the expression of CXCR4, PDGFRa and Flk1 cell surface markers (FIG. 4D). Also the temporality of the appearance of CXCR4/PDGFRa/Flk1 triple positive cells is similar to the temporality of Mesp1-GFP expression (FIG. 4E), suggesting that CXCR4+/PDGFRa+/Flk1+ cells likely represent Mesp1-expressing cells.

To determine whether the combination of PDGFRa, Flk1 and CXCR4 can be used as a substitute to the Mesp1-GFP cells to isolate MCPs, it was shown that the cell population that coexpresses Flk1/PDGFRa and CXCR4 is enriched 3.5 fold for Mesp1 transcript (FIG. 4F). After isolation of the Flk1+/PDGFRa+/CXCR4+ triple positive population at D3 of ESC differentiation and replating for 8 more days, beating areas were restricted to the cells issued from the Flk1+/PDGFRa+/CXCR4+ population, as observed for the Mesp1-GFP positive cells. Quantification of cardiac (FIG. 4G), endothelial (FIG. 4H) and smooth muscle cell markers (FIG. 4I) following terminal differentiation of isolated CXCR4/PDGFRa/Flk1 triple positive cells at D3 revealed that isolated CXCR4/PDGFRa/Flk1 triple positive cells are similarly enriched in early cardiovascular progenitors than the Mesp1-GFP cells at D3 of ESC differentiation (FIGS. 2A-C) and that Mesp1-GFP cells can be substituted by the use of a combined CXCR4/PDGFRa/Flk1 detection to allow the quantification and isolation of early common cardiovascular progenitors for both heart fields, that are able to generate all cardiovascular lineages.

The invention therefore provides tools and methods for identifying and isolating MCPs from e.g. ES cell cultures or EBs, based on the cell surface coexpression of the three markers Flk1, PDGFRa and CXCR4. The present invention thus provides a novel combination of monoclonal antibodies against PDFGRa, CXCR4 and Flk1 that can be used to specifically isolate Mesp1 expressing MCPs, which refine considerably the enrichment of MCPs over the previously published methods by allowing the isolation of early MCPs for both first and second heart field, that are able to generate all cardiovascular lineages after differentiation. This opens very interesting perspectives for providing a simple, reliable and more effective method for isolating these early MCPs, i.e. multipotent cardiovascular progenitors of Day 3 or 4 of ESC differentiation, preferably of Day 3 of ESC differentiation.

Means or binding molecules for detecting the expression of said cell-surface markers are known in the art and examples are for example an antibody, a polypeptide, a peptide, a lipid, a carbohydrate, a nucleic acid, peptide-nucleic acid, small molecule, small organic molecule, or other drug candidate. As used herein, the term antibody includes, but is not limited to, polyclonal antibodies, monoclonal antibodies, humanised or chimeric antibodies, engineered antibodies, and biologically functional antibody fragments (e.g. scFv, nanobodies, Fv, etc) sufficient for binding of the antibody fragment to the protein. Such antibody may be commercially available antibody against its target, such as, for example, a mouse, rat, human or humanised monoclonal antibody. According to an aspect of the invention, a specific binding molecule preferably binds specifically to its target with an affinity better than 10⁻⁶ M.

The invention thus provides kits or tools for isolating or identifying MCPs, preferably from ES cell cultures or EBs, comprising means or binding agents specific for the three markers Flk1, PDGFRa and CXCR4.

Preferably, said binding molecules are detectably labeled for detection and separation by any means of cell sorting apparatus or method including e.g. optical flow cytometry (based on fluorescence detection of a specific binding molecule such as in FACS analysis), magnetic cell separation (using specific binding molecules coated with magnetic particles (MACS)) or density-based cell separation (e.g. using specific binding molecules coated with particles of a certain density). All cell-sorting methods known in the art can be used. For therapeutic use, preferably high throughput sorting methods are applied that can be performed under aseptic conditions.

Magnetic-activated cell sorting (MACS™) is a method for separation of various cell populations depending on their surface antigens (provided by Miltenyi BiotecMagnetic). Magnetic cell separation using target-specific binding molecules is also possible using magnetic beads (e.g. from Dynal/Invitrogen), wherein the magnetic beads with attached cells, protein or nucleic acids are isolated by insertion of the sample tube in a magnetic rack. Also column sorting may be used, which is placed in a strong magnetic field. In this step, the cells expressing the target antigen are attached to the beads linked to a specific binding molecule and stay on the column, while other cells (not expressing the antigen) flow through.

For Fluorescence Activated Cell Sorting (FACS) analysis, the following exemplary fluorescent markers can be used to label the binding molecules, based on the laser light used: Blue argon laser (488 nm): Green: FITC, Alexa Fluor 488, GFP, CFSE, CFDA-SE, DyLight 488; Orange: PE, PI; Red: PerCP, PerCP-Cy5.5, PE-Alexa Fluor 700, PE-Cy5 (TRI-COLOR), PE-Cy5.5; Infra-red: PE-Alexa Fluor 750, PE-Cy7. Red diode laser (635 nm): APC, APC-Cy7, APC-eFluor 780, Alexa Fluor 700, Cy5, Draq-5. Violet laser (405 nm): Pacific Orange, Amine Aqua, Pacific Blue, DAPI, Alexa Fluor 405, eFluor 450, eFluor 605 Nanocrystals, eFluor 625 Nanocrystals, eFluor 650 Nanocrystals.

To further confirm that CXCR4+/PDGFRa+/Flk1+ triple positive cells represent cardiovascular progenitors that mature to cardiovascular lineages during their differentiation, the expression of cardiovascular transcription factors was investigated in CXCR4+/PDGFRa+/Flk1+ triple positive cells over time. RT-PCR analysis performed on FACS-isolated CXCR4+/PDGFRa+/Flk1+ triple positive cells at D3 showed that MCPs isolated using CXCR4/PDGFRa/Flk1 monoclonal antibodies (FIG. 5D) display a similar enrichment for the expression of cardiovascular transcriptional factors as compared to Mesp1-GFP cells (FIG. 5A). In CXCR4+/PDGFRa+/Flk1+ triple positive cells some transcription factors (Hand1, Hand2, Nk×2-5, Gata6 and Tbx20) increased between D3 and D4 (FIG. 4D), suggesting that early specified MCPs coexpressing CXCR4/PDGFRa and Flk1 undergo a progressive maturation toward cardiovascular differentiation over time.

Another object of the invention was the identification of extrinsic factors that stimulate Mesp1 expression and hence MCP specification in order to create a method or tool for the generation of cardiovascular cells or progenitors at large scale, and for the fine comprehension of the earliest steps of cardiovascular development. By investigating the signaling pathways that control the expression of Mesp-1, the inventors showed that Wnt, BMP, Notch, FGF and Nodal pathways are preferentially activated in MCPs. Quantifying Mesp1 expression by flow cytometry, indicated that a proper Wnt, BMP and Nodal activity is required to allow MCPs specification (FIG. 6A). Moreover, using serum free culture system, it was shown that BMP4 and Wnt3a and Wnt5a act in early EBs to induce Mesp1 expression and subsequent MCP specification (FIG. 6B).

The information above enabled inventors to provide tools for identifying and screening agents that influence or promote the differentiation and maturation of ES cells into early MCPs and into cardiovascular cells, which will have very important implications in both the therapeutic field and in research and diagnosis.

One object of the invention is a new Mesp1-reporter gene construct which was used to transform the ES cells as used above. Said construct can be used to follow the development and differentiation of early MCPs common for both first and second heart fields, and to better characterize the cellular and molecular mechanisms regulating early MCP specification. The transformed ES cell line expresses a Venus-GFP reporter under the control of the regulatory region representing the 5.6 kB upstream of the Mesp1 coding sequence (accession number NM_{—}008588), taking the translation start as a reference (FIG. 1A). This sequence faithfully recapitulates Mesp1 expression in transgenic embryos in vivo (Haraguchi et al Dev 2001). The DNA sequence of the complete Mesp1-VenusGFP plasmid construct used is represented by SEQ ID NO.1. The linear fragment between Pacl and BamH1 restriction sites was electroporated in E14Tg2a mouse ES cells to generate the Mesp1-GFP cell line.

The inventors further generated a Mesp1-Luc reporter to screen with a higher throughput the molecules controlling Mesp1 expression. The construct is constructed similarly to the construct using the Venus/GFP reporter gene, wherein the coding sequence for GFP is replaced by the Luciferase coding sequence (cf. SEQ ID NO.2).

The invention thus provides a multipotent cardiovascular progenitor reporter gene-construct (called “MCP reporter construct” hereinafter) and its use for detecting early MCPs common for both first and second heart fields, monitoring the development and differentiation of MCPs and cardiovascular cells derived therefrom.

In a preferred embodiment, the MCP reporter construct according to the invention comprises a reporter gene such as a GFP or Luc. Gene, cloned in the gene regulatory region of the Mesp-1 gene. The MCP reporter construct can additionally comprise a selection marker such as an antibiotics resistance gene, known in the art.

Said selection gene is in preferred embodiments flanked by a recombination site, which enables the excision of the selection gene from the construct. Exemplary site-directed recombination sites are known in the art, e.g. cre-loxP, FRT-FLP, lambda integrase, etc. The inventors here used the Flippase Recognition Target (FRT) sequence, which can be cleaved by the Flippase enzyme. The antibiotic resistance gene can be placed under control of a prokaryotic and/or eukaryotic promoter sequence. The present invention used the pgk/em7 combination of a prokaryotic (em7) and eukaryotic (pgk) promoter to trigger the expression of the neomycin resistance gene.

In a preferred embodiment, the MCP reporter construct is defined by SEQ ID NO. 1 and comprises the Mesp-1 gene regulatory sequence, wherein the GFP-coding sequence is cloned. This part is followed by a neomycin resistance gene, driven by a pgk/em7 promoter duo, flanked by two FRT sites (cf. FIG. 1A and SEQ ID NO.1).

The coding sequence of Mesp-1 is in fact exchanged by the coding sequence of the GFP reporter gene. This has as a result that the endogenous Mesp-1 expression itself is not deregulated at all in the transformed ES cells. Due to the use of the Mesp1 regulatory sequence, the MCP reporter construct follows the Mesp-1 expression pattern and enables visualization of said profile (and thus also the MCP cells expressing it) in real time, without affecting the function of Mesp-1.

The MCP reporter construct of the invention can thus be used for the visualization and detection of early MCPs common for both heart fields, that are able to give rise after differentiation to all cardiovascular lineages.

In addition, the MCP reporter construct of the invention can be used for real-time imaging of the differentiating or developing MCPs.

The latter aspect is interesting for screening or testing agents or drugs for e.g. their toxic or pharmacological effect on the development of and propagation of MCPs.

In addition, the MCP reporter construct can be used for screening factors or agents that stimulate differentiation of MCPs.

Exemplary reporter genes are well known in the art. Non-limiting examples are the following: (enhanced)GFP, venus GFP, (enhanced)CFP, (enhanced)YFP, luciferase, and the like.

Exemplary selection genes are also well known in the art. Non-limiting examples are the following: Neomycin, Ampicillin, Kanamycin resistance genes etc.

The invention further provides for a vector, or plasmid carrying the MCP reporter construct of the present invention.

In addition, the invention provides for a host cell which is transformed with the MCP reporter construct, vector or plasmid of the present invention. Preferably, said host cells are bacteria, in order to provide an easy source of reproducing the construct. Alternatively, said MCP reporter construct can be stably introduced into the genome of a stem cell as defined herein, enabling the visualization of the stem cell development linked to Mesp1 expression, i.e. the cardiovascular development of said stem cells.

In a preferred embodiment of the invention, the cell transformed with the MCP reporter construct is an ES cell-line, stably transformed with said plasmid. Preferably, said ES cell-line is of human origin. In a particularly preferred embodiment, the ES cell-line is the cell-line deposited under the name “Mesp1-GFP” with the Belgian Co-ordinated Collections of Micro-organisms (BCCM/LMBP) under the provisional deposit number LMBP 8051 CB, on Dec. 23, 2010.

The present invention further provides for a stem cell population which is enriched or for cardiovascular progenitor cells, or which is substantially purely comprising MCPs, for example by sorting for cells that express the cell surface markers FlK1, PDGFRa, and CXCR4.

The populations of MCPs contain at least about 30%, and preferably at least about 40%, and more preferably at least about 50% MCPs. In other embodiments, the populations of MCPs contain at least about 70%, and preferably at least about 80% and more preferably at least about 90% and up to 100% MCPs.

The MCPs of the present invention are further characterized in that they are selected at a very early stage of ES cell development or differentiation, i.e. Day 3 or 4, preferably Day3 of said development. The MCP's of the present invention are capable of differentiating into cardiovascular cells of bot the primary and secondary heart field.

The MCPs of the present invention are also useful for generating subpopulations of cardiomyocytes including, for example, atrial, ventricular, and pacemaker cells, using differentiation conditions known to those of skill in the art. In another embodiment, the present invention therefore provides a method of generating cardiovascular colonies containing cardiomyocytes, endothelial cells, and vascular smooth muscle cells. The presence of cardiomyocytes, endothelial cells, and vascular smooth muscle cells can be determined by measuring expression of genes indicative of cardiomyocytes, such as cTNT, and genes indicative of endothelial cells, such as CD31, VE-Cadherin and genes indicative of vascular smooth muscle cells, such as SMA and Calponin.

The present invention further provides methods for screening for agents that have an effect on human MCPs, cardiovascular colonies, cardiomyocytes, endothelial cells and vascular smooth muscle cells. The method comprises contacting ES cells from one of the cell populations described herein with a candidate agent, and determining whether said agent has an effect on the differentiation state or cell fate of the cell population, i.e. whether or not more or less MCPs are present in the culture. The ES cells may be genetically modified so as to express a GFP labeled Mesp-1 protein (GFP or Luc) or may be non-modified ES cells. In the first case, the number of MCPs is determined by Mesp-1 expression, while in the second case, the tools as provided herein for detecting the cell surface markers Flk1, PDGFRa and CXCR4 can be used in order to detect the amount of MCPs formed. The agent to be tested may be natural or synthetic, one compound or a mixture, a small molecule or polymer including polypeptides, polysaccharides, polynucleotides and the like, an antibody or fragment thereof, a compound from a library of natural or synthetic compounds, a compound obtained from rational drug design, a condition such as a cell culture condition, or any agent the effect of which on the cell population may be assessed using assays indicated above or known in the art. The effect on the cell population may additionally be determined by any standard assay for phenotype or activity, including for example an assay for marker expression, receptor binding, contractile activity, electrophysiology, cell viability, survival, morphology, or DNA synthesis or repair.

Candidate agents identified according to the methods of the invention will be useful for the control of cell growth, differentiation and survival in vivo and in vitro of MCPs and cardiovascular cells and their use in e.g. cardiac or vascular tissue maintenance, regeneration and repair.

The present invention further provides compositions comprising populations of human MCPs and compositions comprising populations of human cardiovascular colonies. Said compositions may comprise pharmaceutically acceptable carriers and diluents. The compositions may further comprise components that facilitate engraftment. Compositions comprising such cell populations are useful for cell and tissue replacement and repair, and for generating populations of cardiomyocytes, endothelial cells, and vascular smooth muscle cells in vitro and in vivo. Compositions comprising human MCPs are useful for expansion of the progenitor populations. The compositions may be formulated as a medicament or delivery device for treating a cardiac condition.

In another embodiment, the present invention provides methods of cell replacement and methods of tissue replacement useful for treatment of disorders characterized by insufficient cardiac function including, for example, congenital heart disease, coronary heart disease, cardiomyopathy, endocarditis and congestive heart failure.

Both the differentiated cells and the cardiovascular progenitor cells are useful for replacement therapy, since the progenitor populations are capable of differentiation to the cardiomyocyte, endothelial and vascular smooth muscle lineages in vivo. The cells are also useful for generating cardiovascular tissue in vitro.

Methods for engineering cardiac tissue are known in the art and reviewed for example by Birla in “Stem Cell Therapy and Tissue Engineering for Cardiovascular Repair” Springer, 2006.

Accordingly, in one embodiment the present invention provides a method of cardiomyocyte replacement therapy comprising administering to a subject in need of such treatment a composition comprising MCPs obtained by the methods of the present invention, or cardiovascular cells obtained therefrom.

In another embodiment, the present invention provides a method of treating a disorder characterized by insufficient cardiac function comprising administering to a subject in need of such treatment a composition comprising MCPs obtained by the methods of the present invention, or cardiovascular cells obtained therefrom.

In a preferred embodiment, the subject is a human.

The composition may be administered by a route that results in delivery to or migration to cardiac tissue including, for example, injection, grafting or implantation, and under conditions that result in a reduction of at least one adverse effect or symptom or the disorder.

The MCPs according to the invention are particularly useful in medicine, cardiology, inborn errors of hearty functioning or differentiation, transplantation, infectious diseases, heart failure. The heart stem cells according to the invention are particularly useful for (human) heart cell transplantation, the preparation of animal models of human heart cell transplantation, bioartificial hearts, in vitro heart cell lines and animal models of acquired human heart diseases or heart-function disorders, heart rhythm tests and heart cell directed gene therapy. The heart stem cell according to the invention can be further differentiated into cardiomyocytes or vascular cells.

Preferably, the invention provides a differentiated cell produced using methods of the invention that may be used for therapeutic purposes, such as in methods of restoring cardiac function in a subject suffering from a heart or vascular disease or condition.

The invention thus provides a method of treating or preventing a cardiovascular disease or condition. Cardiac disease is typically associated with decreased cardiac function and includes conditions such as, but not limited to, myocardial infarction, cardiac hypertrophy and cardiac arrhythmia. In this aspect of the invention, the method includes introducing an isolated differentiated cardiomyocyte cell of the invention and for a cell capable of differentiating into a cardiomyocyte cell when treated using a method of the invention into cardiac tissue of a subject. The isolated cardiomyocyte cell is preferably transplanted into damaged cardiac tissue of a subject. More preferably, the method results in the restoration of cardiac function in person suffering from chronic or acute cardiac insufficiency.

In yet another aspect of the invention there is provided a method of repairing cardiac tissue, the method including introducing an isolated cardiomyocyte or cardiac progenitor cell of the invention and for a cell capable of differentiating into a cardiomyocyte cell when treated using a method of the invention into damaged cardiac tissue of a subject.

The present invention also provides a source of cardiovascular cells for tissue engineering, that can be used in the development of transplantation therapies and for research purposes.

The methods of the invention further allow a large production of cardiovascular cells, which is a substrate of choice for the large scale screening of new molecules, or identification of novel effects of known drugs in cardiovascular drug research.

The present invention also provides a method of conducting in vitro drug metabolism studies comprising: (i) exposing a heart stem cell or cell population thereof according to the invention, to a test agent, and (ii) observing at least one change, if any, involving the test agent after a predetermined test period.

The invention further provides for an assay for assessing the toxicity of an agent on heart or vascular cells, comprising the steps of: a) differentiating stem cells into cardiovascular progenitor cells according to the method of the invention, b) subjecting said cells in vitro to said agent, and c) analysing the toxic effect of said agent on the cells obtained in step a). So far, no method is available to generate specific cardiovascular cells. The present invention allows the generation of large source of cardiovascular cells that can be used to perform a large scale screening of drug toxicity or to study the molecular mechanism underlying cardiac toxicity of drugs and to identify new targets to prevent it. The present invention also provides a method of conducting in vitro toxicity testing comprising: exposing to a test agent a heart stem cell or cell population thereof according to the invention, and observing at least one effect, if any, of the test agent on the population of heart cells. Preferably, the at least one effect includes an effect on cell viability, cell function, or both.

The Mesp1 expressing cells as defined herein were subsequently used for the identification of novel biomarkers of MCPs and for better characterization of the early molecular events occurring in during MCP specification. To this end, the expression profiles of Mesp1-GFP expressing cells (GFP-positive) with those of Mesp-1-negative cells (GFP-negative) at D3 of ESC differentiation were compared by microarray analysis. We determined which genes displayed a change in expression of at least 1.5 fold. Using these criteria, Mesp1 expression was associated with a change in expression of 1151 probes (for 45101 screened probes), representing a change in expression of 2.5% of the murine genome. Among the 212 upregulated genes, we identified several genes involved in early mesodermal and cardiovascular development that are enriched in the Mesp1-GFP fraction.

Interestingly, concomitant expression of Mesp1 and Isl1 in early cardiovascular progenitors at D3 was observed, which is consistent with the notion that Mesp1 mark early progenitor of both primary and second cardiac heart fields, and suggests that the early Mesp1 expressing cells represent a primitive multipotent Mesp1/Isl1 cardiovascular progenitor during ESC differentiation (Laugwitz et al., 2008, Development 135, 193-205). We confirmed by immunofuorescence that isl1 is indeed coexpressed with Mesp1 in differentiating EBs at D3 in a subpopulation of Mesp1-expressing cells (FIG. 3), consistent with the isolation of a common precursor for the primary and second heart field during ESC differentiation using Mesp1-GFP cells.

Many Transcription factors and proteins involved in signaling pathways were also found to be induced by Mesp-1 expression. Examples are e.g.: transcription factors: Hoxb1, Wnt5a, Foxf1a, Isl1, Hoxb2, Etv2, Tbx3, Snai1, Tbx6, Prrx2, Smarcd3, Msx2, Mesp2, Tbx2, Hand1, Meis2, Tbx3, Evx1, Six2, Gata4, LOC100046086, Tbx20, Tbx3, Bhlhe22, Hoxd1, Lef1, Msx1, Zfp423, Ets1, Cbx4, Zbtb44, Hmga2, and/or any other transcription factor; pathway involved proteins: Smad1, Fgf10, Fgf15, Wnt3, Bambi, Braf, Fgf3, D113, Bmp4, Wnt5b, Dll1, Ptgds, Wnt2, Wnt5a and/or any other pathway involved protein.

Furthermore, Ets1 and Etsv2 are also enriched in Mesp1 expressing cells. By cooperating with the Fox family of transcription factors, Ets transcription factors have been shown to control endothelial development (De Val et al., 2008, Cell 135, 1053-64). Our data identified a subset of transcription factors that control endothelial and smooth muscle development within MCPs and that cooperate to organize vascular differentiation of MCPs.

We also found that early cardiovascular progenitors preferentially express genes that are involved in epithelial to mesenchymal transition (EMT) such as Snail1, Foxc1, Foxc2, Twist1 and Twist2 (FIG. 5B) (Kalluri et al., 2009, J Clin Invest 119, 1420-8). We identified that EMT is one of the earliest event leading to cardiovascular development and MCPs differentiation (FIG. 5C). This EMT activity is controlled by specialized transcription factors and leads to the acquisition of mesenchymal properties to the developing cardiovascular cells.

The microarray-based transcriptional profiling of Mesp1-GFP cells as performed herein demonstrated that MCPs preferentially expressed some 20 different cell surface markers, such as: Pcdh19, Ceacam10, Adcyap1r1, Htr1d, Cmklr1, S1pr5, Pmp22, Adrb1, Kdr, Vldlr, Pcdh18, Il13ra1, Cd160, L1cam, Cxcr4, Cxcr7, Gpr177, Ednra, Pdgfrb, Cdh11, Vstm2b, Pcdh7, Tmem88, Apinr, Pgr, Lrp1, Cdh2, Nrp1, Antxr1 and/or any other transmembrane receptor. Up to now, some markers have been used at later stages of ES cell differentiation (D5-D6) to isolate specialized subpopulations of cardiovascular progenitors such as progenitors of the first heart field progenitors (Flk1/CXCR4-Nelson et al. Stem Cells 2008), and second heart field progenitors (Moretti et al. Cell 2006), as well as only bipotent progenitors for cardiac and smooth muscle cells (PrP and Flk1—Hidaka et al Circ Res 2010). The invention presents here a novel combination of monoclonal antibodies against PDFGRa, CXCR4 and Flk1 that can be used to specifically isolate Mesp1 expressing early MCPs, which refine considerably the enrichment of MCPs over the previously published methods, allows the isolation of early cardiovascular progenitors for both heart fields that are able to give rise after differentiation to all cardiovascular lineages, and opens very interesting perspectives for providing a simple, reliable and more effective method for isolating MCPs.

The invention thus provides tools and methods for isolating, identifying or monitoring formation of MCPs, preferably from ES cell cultures or EBs, based on the expression of genes, regulated by the Mesp-1 expression. In a preferred embodiment, the markers Flk1, PDGFRa and CXCR4 can be used for isolating MCPs out of a differentiating culture of ES cells.

To identify the role of transcription factors that are coexpressed with Mesp1 in MCP specification and cardiovascular differentiation of EBs, ESC lines were generated that allow a forced expression of genes that we found to be coexpressed in Mesp1 expressing cells, alone or in combination with forced Mesp1 expression. Among the tested candidate genes we identified that a forced co-expression of Mesp1 and Hey2, a central effector of Notch signaling during cardiovascular development which is preferentially expressed in ventricular cells (High et al., 2008, Nat Rev Genet 9, 49-61), induced a massive generation of MCPs that coexpress Flk1/PDGFRa and CXCR4, while a forced expression of Hey2 alone does not (FIGS. 7B and 7C). Also Mesp1 and Hey2 co-expression was found to be further increasing the promotion of EMT compared to Mesp1 alone (FIGS. 8B and 8C). The gain of function of Hey2 alone from D2 to D4 did not increase the proportion of CMs measured at D8 (FIGS. 7D and 7E), while it resulted in a marked increase in EC differentiation (FIGS. 7F and 7G). In sharp contrast, co-expression of Mesp1 and Hey2 resulted in massive differentiation of ESCs into both cardiac and vascular cells, to a larger extent than the cardiovascular promoting effect of Mesp1. By RT-PCR analysis, it was shown that co-expression of Mesp1 and Hey2 resulted in a stronger increase of TropT2, aMHC, MIc2a and MIc2c, Tbx5, Tbx18 and Wt1 than following Mesp1 expression alone (FIG. 7H), suggesting that Mesp1 and Hey2 cooperate to promote different cardiac lineages commitment during ESC differentiation. To investigate the molecular mechanism by which Mesp1 and Hey2 cooperate during MCP specification and cardiac lineage commitment, the expression of the cardiovascular and EMT transcription factors following Hey2, Mesp1 and Mesp1/Hey2 expression 48 h following Dox addition was analyzed by RT-PCR. It was shown that Hand2, Nk×2-5, Tbx20, FoxF1a and Twist1 were more upregulated following Mesp1 and Hey2 co-expression compared to Mesp1 or Hey2 alone (FIGS. 71 and 8B), which was accompanied by a stronger EMT compared to Mesp1 alone (FIG. 8C). These data showed that Notch activity cooperate with Mesp1 functions in promoting the specification of MCPs and that Notch can promote the differentiation of MCPs to ventricular cells.

Signaling pathways that control the expression of Mesp1 are not yet identified. In addition, as Mesp1 acts as a master regulatory switch during cardiovascular development once its expression is transiently activated, the identification of pathways that control Mesp1 expression is an important prerequisite that could offer a new tool for the generation of cardiovascular cells or progenitors at large scale, and for the fine comprehension of the earliest steps of cardiovascular development.

The invention provides tools for identifying and screening agents that can influence the differentiation and maturation of ES cells into MCPs and into cardiovascular cells, which will have very important implications in both the therapeutic field and in research and diagnosis.

In addition, a Mesp1-Luc reporter system was used to screen the molecules controlling Mesp1 expression with a higher throughput.

The present invention further provides methods for screening for agents that have an effect on human MCPs, cardiovascular colonies, cardiomyocytes, endothelial cells and vascular smooth muscle cells. The method comprises contacting ES cells from one of the cell populations described herein with a candidate agent, and determining whether said agent has an effect on the differentiation state or cell fate of the cell population, i.e. whether or not more or less MCPs are present in the culture. The ES cells may be genetically modified so as to express a GFP labeled Mesp-1 protein (GFP or Luc) or may be non-modified ES cells. In the first case, the number of MCPs is determined by Mesp-1 expression, while in the second case, the tools as provided herein for detecting the cell surface markers Flk1, PDGFRa and CXCR4 can be used in order to detect the amount of MCPs formed. The agent to be tested may be natural or synthetic, one compound or a mixture, a small molecule or polymer including polypeptides, polysaccharides, polynucleotides and the like, an antibody or fragment thereof, a compound from a library of natural or synthetic compounds, a compound obtained from rational drug design, a condition such as a cell culture condition, or any agent the effect of which on the cell population may be assessed using assays indicated above or known in the art. The effect on the cell population may additionally be determined by any standard assay for phenotype or activity, including for example an assay for marker expression, receptor binding, contractile activity, electrophysiology, cell viability, survival, morphology, or DNA synthesis or repair.

Candidate agents identified according to the methods of the invention will be useful for the control of cell growth, differentiation and survival in vivo and in vitro of MCPs and cardiovascular cells and their use in e.g. cardiac or vascular tissue maintenance, regeneration and repair.

The present invention has important implications for these clinical applications, in which increasing the efficiency of cardiovascular differentiation would be needed to be useful in practice. By temporally regulating the expression of Mesp1-co-expressed genes or genes belonging to pathways upstream of Mesp-1 expression, the invention provides a method to produce high amount of cardiovascular cells that could be transplanted in patients or animals suffering from any condition where cardiac, vascular or conductive cells are lacking.

The invention further provides for a method for performing cellular therapy, comprising the steps of: a) providing cells according to the method of the invention, b) specifying and differentiating the cardiovascular progenitors generated by method of the invention into a particular subset of cardiovascular lineages such as cardiomyocytes, vascular or endothelial cells and c) injecting said cells into the heart or the vasculature of the subject in need thereof allowing exogenous, autologous or not, cell therapy.

The present invention opens new perspectives in the recruitment, amplification, migration and differentiation processes of these multipotent endogenous progenitors following cardiac injury. In such embodiment, the invention further provides for method for restoring the heart or vasculature function in an endogenous manner, in a subject in need thereof, comprising the step of transiently inducing the expression of genes regulating the Mesp-1 expression. Preferably, said induction is performed by injecting the subject with an amount of an expression vector encoding for the Mesp-1 regulator selected from the group comprising Flk1, PDGFRa and CXCR4. Alternatively, said induction is performed by injecting a factor or an agent inducing the expression of any one or more of said Mesp-1 regulatory genes in said cells of the heart or the vasculature.

To uncover the molecular mechanisms by which Mesp1 induced cardiovascular specification, the inventors performed a genome wide analysis of Mesp1 regulated genes. The inventors determined which genes were regulated upon Mesp1 induction. In this embodiment, the invention leads to a prospective identification, quantification and characterization of the cardiovascular potential of any isolated cell for cardiovascular cell therapy by analyzing the expression pattern of one or more genes selected from the group comprising Flk1, PDGFRa and CXCR4.

The methods of the invention further allow a large production of MCPs or cardiovascular cells, which is a substrate of choice for the large scale screening of new molecules, or identification of novel effects of known drugs in cardiovascular drug research.

The invention further provides for use of MCPs or cardiovascular cells obtained by the methods as indicated above, for evaluating the cardiovascular effects of a drug on differentiated cardiac cells or for evaluating the cardiovascular effects of a drug during cardiovascular development. The invention provides for an assay for assessing the pharmacology of a candidate drug comprising the steps of: a) differentiating stem cells into cardiovascular progenitor cells according to the method of the invention, b) subjecting said cells in vitro to said candidate drug, and c) analysing the behaviour of said cells in the presence and absence of said candidate drug.

In another embodiment, the invention further provides for use of MCPs or cardiovascular cells obtained by the methods as indicated above for the preparation of a medicament for restoring cardiovascular functioning in a subject.

The present invention also provides a method of conducting in vitro drug metabolism studies comprising: (i) exposing an MCP population according to the invention, to a test agent, and (ii) observing at least one change, if any, involving the test agent after a predetermined test period.

The invention further provides for an assay for assessing the toxicity of an agent on heart or vascular cells, comprising the steps of: a) differentiating stem cells into cardiovascular progenitor cells according to the method of the invention, b) subjecting said cells in vitro to said agent, and c) analysing the toxic effect of said agent on the cells obtained in step a).

So far, no method is available to generate specific cardiovascular cells. The present invention allows the generation of large source of cardiovascular cells that can be used to perform a large scale screening of drug toxicity or to study the molecular mechanism underlying cardiac toxicity of drugs and to identify new targets to prevent it. The present invention also provides a method of conducting in vitro toxicity testing comprising: exposing a test agent to an MCP cell population according to the invention, and observing at least one effect, if any, of the test agent on the population of MCP cells. Preferably, the at least one effect includes an effect on cell viability, cell function, differentiation or both.

The invention further provides for tools for molecular diagnosis in Congenital Heart Disease (CHD). Different congenital heart diseases are characterized by abnormal closure or malposition of cardiac structures following a migration or specification defect. The invention therefore further encompasses a method for studying genetic defects in Mesp1 during the onset of progression of cardiac malformation. The occurrence of mutation of in genes regulated by Mesp1 expression (i.e. one or more genes selected from the group comprising Flk1, PDGFRa and CXCR4) in congenital heart diseases, in which a cell migration or specification defect can be involved, can also be studied. The invention provides a large scale strategy that will allow the clinical detection of such condition. The present invention also provides a method for enhancing the regeneration of an injured or diseased heart comprising administering into the liver an effective amount of a heart stem cell or cell population thereof according to the invention.

The present invention also provides a method for treating errors of gene expression comprising: (i) introducing into an MCP cell population prepared according to the invention a functional copy of one or more genes selected from the group comprising Flk1, PDGFRa and CXCR4, to provide a transformed population; and (ii) introducing into a patient's heart, which patient is in need of the functional copy of the gene, at least a portion of the transformed population.

The present invention also provides a composition for treating errors of gene expression comprising a transformed heart stem cell or cell population thereof according to the invention into which a functional copy of one or more genes selected from the group comprising Flk1, PDGFRa and CXCR4 has been introduced.

The present invention also provides a pharmaceutical composition for treating errors of gene expression comprising a heart stem cell or cell population thereof prepared according to the invention into which a functional copy of one or more genes selected from the group comprising Flk1, PDGFRa and CXCR4 has been introduced and a pharmaceutically acceptable carrier.

### EXAMPLES

The invention is illustrated by the following non-limiting examples

### Materials and Methods

### ES Cells Culture and Differentiation

ESCs were cultured on irradiated MEFs in DMEM supplemented with 15% ESC-qualified FBS (Gibco), 0.1 mM non essential amino acids (Gibco), 1 mM sodium-pyruvate (Gibco), 0.1 mM 13-mercaptoethanol (Sigma), 100 U/ml Penicillin (Gibco), 100 μg/ml Streptomycin (Gibco) and 1000 μml LIF (ESGRO). ESC differentiation was performed in hanging drops of 1000 cells in 25 μl, as previously described (Bondue et al., 2008). To assess the cardiovascular potential of Mesp1-GFP and CXCR4/PDGFRa/Flk1 TP cells, EBs were cultured for 3 days in hanging drops in differentiation medium consisting of the same medium without LIF, but containing 15% of ESC-qualified serum (Invitrogen) and ascorbic acid (50 μg/ml, Sigma) (Bondue et al., 2008). At D3, dissociated cells were stained and sorted in HBSS containing 2% FBS, washed and replated on gelatin-coated dishes in a serum-free medium based on StemPro34 (Invitrogen), supplemented with 100 U/ml Penicillin (Gibco), 100 μg/ml Streptomycin (Gibco), L-Glut (2 mM), ascorbic acid (50 μg/ml, Sigma), b-FGF (10 ng/ml), FGF10 (25 ng/ml), VEGF (5 ng/ml), PDGFRa (100 ng/ml), and hDKK1(150 ng/ml) (Kattman et al., 2006). All growth factors were purchased from R&D. Medium was replaced on days 5, 7 and 9 of differentiation. For low density culture assays, 50 isolated cells were replated in each well of 8-well Labtek glass chamber slides (Nunc), with Y-27632 (Calbiochem) at a final concentration of 10 μM for the first 48 hours. Dox-inducible ESC lines were differentiated in DMEM containing 15% ESC-qualified serum and ascorbic acid (50 μg/ml, Sigma). After four days in hanging drops, embryoïd bodies were replated on gelatin-coated dishes for further differentiation. Dox (Sigma) was added to hanging drops at corresponding days to a final concentration of 1 μg/ml as previously described (Bondue et al., 2008). Inhibition of signaling pathways were performed in a serum containing medium using Dkk1 (300 ng/ml) and Noggin (200 ng/ml) obtained from R&D, or using SB431542 obtained from Sigma.

### Reverse Transcription and Quantitative PCR

Total RNA extraction and Dnase treatment of samples were performed using Absolutely RNA-microprep kit (Stratagene), according manufacturer's recommendations. 1 μg of purified RNA was used to synthesize the first strand cDNA in a 50 μl final volume, using a SuperscriptII (Invitrogen) and random hexamers (Roche). Control of genomic contamination was performed for each sample by performing the same procedure with or without reverse transcriptase. qPCR analysis was performed with one-twentieth of the cDNA reaction as template, using a Quantifast SYBR Green mix (Qiagen) on a ABI Fast7500 Real-Time PCR system. Analysis of results was performed by using the qBase (Hellemans et al., 2007) and GraphPad Prism software.

### FACS Analysis and Cell Sorting

FACS analysis was performed using a fluorescence-activated cell analyser (FACSCanto, Beckton Dickinson, Immunocytometry Systems); cell sorting using a fluorescence-activated cell sorter. Data were analyzed using BDFacsDiva software (Beckton Dickinson, Immunocytometry Systems). Following antibodies were used: PDGFRa-PE (1/75; eBiosciences), Flk1-Biotin (1/100; eBiosciences), CXCR4-APC (1/100; eBiosciences), and Isl1 (clone 39.4D5 obtained from DSHB).

### Example 1

### Isolation and Functional Characterization of Multipotent Cardiovascular Progenitors Using Mesp1-GFP Reporter ESC Line

To better characterize the cellular and molecular mechanisms regulating MCP specification, we generated an ES cell line that expresses a Venus-GFP reporter under the control of the 5.6 kb genomic region upstream of Mesp1 coding sequence (FIG. 1A), which is known to recapitulate endogenous Mesp1 expression in vivo (Haraguchi et al., 2001, Mech Dev 108, 59-69). The DNA sequence of the complete construct used is represented by SEQ ID NO. 1.

We subsequently electroporated this Mesp1 reporter construct into ES cells and selected neomycin resistant clones. We selected three clones displaying a temporally regulated expression of the Venus-GFP transgene, consistent with the temporal expression of Mesp1 mRNA, which starts to be expressed at D2, peaks at D3 and is followed by a rapid extinction (FIGS. 1B, 1C and 1D) (Bondue et al., 2008, Cell Stem Cell 3, 69-84). No GFP positive cell was observed in undifferentiated ES cells. Mesp1-GFP positive cells appeared between D2 and D3 during ESC differentiation, and at D3, 2.6% of differentiating ESCs expressed the GFP transgene (FIG. 1D). After D4, the percentage of GFP positive cells rapidly decreased (FIG. 1D). This “Mesp1-GFP” cell line was deposited on Dec. 23, 2010 with the Belgian Co-ordinated Collections of Micro-organisms (BCCM/LMBP) under the provisional deposit number LMBP 80510B.

To verify that our Mesp1-GFP reporter ESC line faithfully recapitulated the endogenous Mesp1 expression, we isolated Mesp1-GFP expressing cells by cell sorting at D3, at the peak of GFP expression during ESC differentiation. Using RT-PCR analysis, we found that Mesp1 transcript is enriched by about 5 fold in the Mesp1-GFP positive cells (FIG. 1E). To evaluate the enrichment in cardiac progenitors, we compared the beating potential of these Mesp1-GFP and Mesp1 negative fractions by plating sorted cells at D3 on gelatin coated dishes, in a serum-free medium containing bFGF (10 ng/ml), FGF10 (25 ng/ml), hDKK1 (150 ng/ml), VEGF (5 ng/ml) and PDGF-AA (100 ng/ml) (Kattman et al., 2006, Dev Cell 11, 723-32). After 8 days of incubation, we could detect beating areas only in the GFP positive fraction, but not in the GFP negative cells, suggesting that the cardiac progenitor cells were located exclusively in the Mesp1-GFP expressing cells. To quantify this cardiac enrichment, we measured the expression of a cardiac isoform of troponinT (cTNT) by FACS and showed that the isolation of Mesp1-GFP cells allowed a 7 fold enrichment in cardiac cells, showing that Mesp1-GFP cells represent cardiac progenitors (FIG. 2A). The Mesp1-GFP cells also displayed a greater potential for endothelial and smooth muscle cell differentiation (FIGS. 2B and 2C). By RT-PCR, we also showed that Mesp1 expressing cells can differentiate into the various cell types of the cardiovascular lineage such as atrial cells (MLC2A), ventricular cells (MIc2v, Tbx5), conduction and pacemaker cells (KcnE1) (FIG. 2D). To determine whether Mesp1-expressing cells represent common progenitors for both primary and second heart fields, clonal analysis of Mesp1-expressing cells isolated at D3 of ESC differentiation was performed, after which they were replated at low density to identify the different cell fates that can be generated from the differentiation of single Mesp1-GFP expressing cells. First heart field progenitors are bipotent progenitors that can give rise to cardiac and smooth muscle cells after differentiation (Wu et al. Cell 2006), while second heart field progenitors are tripotent progenitors and are able to generate cardiac, endothelial and smooth muscle cell fates following their differentiation (Moretti et al. Cell 2006, Bu et al. Nature 2009). After differentiation of Mesp1-GFP expressing cells isolated at D3, immunostaining of individual colonies was performed, showing that almost all colonies contained SMA positive cells. About 15% of the clones presented both cardiac and vascular cells, 40% only expressed cTNT and 40% only expressed VE-cadherin (FIGS. 2E and 2F). To determine whether derivatives of both the first and second heart fields are present within the tripotent colonies, single Mesp1-expressing cells were replated and RT-PCR was performed after their differentiation. Similarly to the results obtained by immunostaining, the vast majority of the colonies expressed SMA, among them some colonies also expressed EC or CM markers and some colonies expressed markers of all three lineages, and Tbx5 and Isl1 were both expressed in 40% of the tripotent colonies (FIG. 2G), supporting that a fraction of Mesp1-expressing cells represents common progenitors for both heart fields.

Isolation of Mesp1-GFP-expressing cardiovascular progenitors at D3 of ESC differentiation, followed by their transplantation under the kidney capsule of NOD/SCID mice allowed to identify the cardiovascular potential of Mesp1-GFP MCPs in vivo. Four weeks after transplantation of Mesp1-GFP expressing cells, no teratomas were observed, while Mesp1-GFP negative cells grafted under the other kidney capsule of the heterolateral kidney as control generated teratomas. Immunostainings of the grafts demonstrated that Mesp1-GFP cells mainly differentiated into cardiomyocytes, although expression of endothelial and smooth muscle cell markers were also present within the graft (FIG. 2H).

Isl1 expression has been previously used to mark tripotent MCPs at D5 of ESC differentiation that could represent second heart field progenitors (Moretti et al., 2006). The microarray-based transcriptional profiling of Mesp1-GFP cells as performed herein demonstrated that MCPs preferentially expressed is 11 (FIG. 5A). To better characterize the relation between Mesp1 and Isl1 expression, immunostainings for Isl1 and GFP expression were performed on Mesp1-GFP cells at D3 and D4 following ESC differentiation. By immunofluorescence, Mesp1-GFP was expressed in 4% and 1.5% of cells respectively at D3 and D4 (FIG. 3A). Isl1 expression was lower at D3 than at D4 and that in later stages of differentiation, but could already be detected at the edge of EBs in about 10% of ESCs at D3 and D4 (FIG. 3B). At D3, about 20% of Mesp1-expressing cells co-expressed Isl1 (FIGS. 3C and 3E), while at D4 about 50% of Mesp1-expressing cells co-expressed Isl1 (FIGS. 3D and 3E). The Mesp1/Isl1 double positive cells represented 10% and 6% of Isl1-expressing cells at D3 and D4 of ESC differentiation respectively (FIG. 3F). These observations support the notion that Isl1 a marker tripotent cardiovascular progenitors is expressed together with Mesp1 only in a fraction of early Mesp1-expressing cells, showing that Mesp1 expressing cells represent a common pool of cells for both previously described bipotent (first heart field) and tripotent (second heart field) cardiovascular progenitors. These observations showed that Mesp1 reporter temporally recapitulates endogenous Mesp1 expression and allows the isolation of Mesp1 expressing cells, which correspond to the earliest MCPs, common for all cardiovascular lineages that are able to generate all cardiovascular lineage after their differentiation.

### Example 2

### Molecular Profiling of Early MCPs

To identify novel biomarkers of MCPs and better characterize the early molecular events occurring in Mesp1 expressing cells during MCP specification, we compared by microarray the expression profiles of Mesp1-GFP expressing cells to the GFP negative cells at D3 of ESC differentiation. We determined which genes displayed a change in expression of at least 1.5 fold. Using these criteria, Mesp1 expression was associated with a change in expression of 1151 probes (for 45101 screened probes), representing a change in expression of 2.5% of the murine genome. Among the 212 upregulated genes, we identified several genes involved in early mesodermal and cardiovascular development that are enriched in the Mesp1-GFP fraction.

Although the existence of MCPs is now well documented, the existence of a common progenitor for primary and second heart field remains unclear and a better knowledge of the molecular mechanisms leading to their specification represents an important biological question (Laugwitz et al., 2008, Development 135, 193-205). We identified a concomitant expression of Mesp1 and Isl1 in early cardiovascular progenitors at D3, which is consistent with the notion that Mesp1 mark early progenitor of both primary and second cardiac heart fields, and suggests that the early Mesp1 expressing cells represent a primitive multipotent Mesp1/Isl1 cardiovascular progenitor during ESC differentiation, with heterogeneity within Mesp1 field for Isl1 expression (Laugwitz et al., 2008, Development 135, 193-205; Stolfi et al. Science 2010). We confirmed by immunofuorescence that isl1 is coexpressed with Mesp1 in differentiating EBs at D3 (FIG. 3). Ets1 and Etsv2 are also enriched in Mesp1 expressing cells. By cooperating with the Fox family of transcription factors, Ets transcription factors have been shown to control endothelial development (De Val et al., 2008, Cell 135, 1053-64). Our data identified a subset of transcription factors that control endothelial and smooth muscle development within MCPs and that cooperate to organize vascular differentiation of MCPs.

We also found that early cardiovascular progenitors preferentially express genes that are involved in epithelial to mesenchymal transition (EMT) such as Snail1, Foxc1, Foxc2, Twist1 and Twist2 (Kalluri et al., 2009, J Clin Invest 119, 1420-8). We identified that EMT is one of the earliest event leading to cardiovascular development and MCPs differentiation (FIGS. 5B and 5C). This EMT activity is controlled by specialized transcription factors and leads to the acquisition of mesenchymal properties to the developing cardiovascular cells.

### Example 3

### MCP Isolation Using Flk1/PDGFRa and CXCR4 Monoclonal Antibodies

One of the main challenges in the field would be the development of simple and reproducible approach to isolate MCP using monoclonal antibodies. Our microarray-based transcriptional profiling of Mesp1-GFP cells demonstrated that MCPs preferentially expressed different cell surface markers (FIG. 3A). Among those, we could show that PDFGRa, CXCR4 and Flk1 could be used in combination to specifically isolate Mesp1 expressing MCPs. At the time of MCP specification (D3), the majority of Mesp1-GFP cells (40%) co-expressed both Flk1, PDGFRa and CXCR4 (Flk1+/PDGFRa+/CXCR4+), and a smaller population expressed only PDGFRa (FIG. 4D). To determine whether the combination of PDGFRa, flk1 and CXCR4 can be used as a substitute to the Mesp1-GFP cells to isolate MCPs, we first showed that the cell population that coexpress Flk1/PDGFRa and CXCR4 is enriched 3.5 fold for Mesp1 transcript (FIG. 4F). After isolation of the Flk1+/PDGFRa+/CXCR4 population at D3, we replated these cells in a serum-free medium, as previously done for Mesp1-GFP cells. 8 days after replating, we identified beating areas that were restricted to the cells issued from the Flk1+/PDGFRa+/CXCR4+ population. We showed that this population give rise to a similar enrichment of cardiovascular cells, as we previously observed for the Mesp1-GFP fraction of cells (FIG. 2A-C), showing that the cell population that co-expressed Flk1/PDGFRa and CXCR4 at D3 represent MCPs (FIG. 2A-C).

### Example 4

### Coexpression of Mesp1 and Hey2 Convert All ESC into Ventricular MCPs

To identify the role of transcription factors that are coexpressed with Mesp1 in MCP specification and cardiovascular differentiation of EBs, ESC lines were generated that allow a forced expression of genes that we found to be coexpressed in Mesp1 expressing cells, alone or in combination with forced Mesp1 expression. Among the tested candidate genes we identified that a forced co-expression of Mesp1 and Hey2, a central effector of Notch signaling during cardiovascular development which is preferentially expressed in ventricular cells (High et al., 2008, Nat Rev Genet 9, 49-61), induced a massive generation of MCPs that coexpress Flk1/PDGFRa and CXCR4, while a forced expression of Hey2 alone does not (FIGS. 7B and 7C). Also, Mesp1 and Hey2 co-expression was shown to further increase the promotion of EMT compared to Mesp1 alone (FIGS. 8B and 8C). The gain of function of Hey2 alone from D2 to D4 did not increase the proportion of CMs measured at D8 (FIGS. 7D and 7E), while it resulted in a marked increase in EC differentiation (FIGS. 7F and 7G). In sharp contrast, co-expression of Mesp1 and Hey2 resulted in massive differentiation of ESCs into both cardiac and vascular cells, to a larger extent than the cardiovascular promoting effect of Mesp1. By RT-PCR analysis, it was shown that co-expression of Mesp1 and Hey2 resulted in a stronger increase of TropT2, aMHC, MIc2a and MIc2c, Tbx5, Tbx18 and Wt1 than following Mesp1 expression alone (FIG. 7H), suggesting that Mesp1 and Hey2 cooperate to promote different cardiac lineages commitment during ESC differentiation. To investigate the molecular mechanism by which Mesp1 and Hey2 cooperate during MCP specification and cardiac lineage commitment, the expression of the cardiovascular and EMT transcription factors following Hey2, Mesp1 and Mesp1/Hey2 expression 48 h following Dox addition was analyzed by RT-PCR. lit was found that Hand2, Nk×2-5, Tbx20, FoxF1a and Twist1 were more upregulated following Mesp1 and Hey2 co-expression compared to Mesp1 or Hey2 alone (FIGS. 71 and 8B), which was accompanied by a stronger EMT compared to Mesp1 alone (FIG. 8C). These data showed that Notch activity cooperate with Mesp1 functions in promoting the specification of MCPs and that Notch can promote the differentiation of MCPs to ventricular cells.

### Example 5

### Identification of Molecules that Stimulate Mep1 Expression and MCP Specification

Signaling pathways that control the expression of Mesp1 are not yet identified. In addition, as Mesp1 acts as a master regulatory switch during cardiovascular development once its expression is transiently activated, the identification of pathways that control Mesp1 expression is an important prerequisite that could offer a new tool for the generation of cardiovascular at large scale, and for the fine comprehension of the earliest steps of cardiovascular development. Based on our molecular profiling data, we identified that Wnt, BMP, Notch, FGF and Nodal pathways are preferentially activated in MCPs. Quantifying Mesp1 expression by flow cytometry, we characterized that a proper Wnt, BMP and Nodal activity is required to allow MCPs specification (FIG. 6A). Moreover, using serum free culture system, we identified that BMP4 and Wnt3a and Wnt5a act in early EBs to induce Mesp1 expression and subsequent MCP specification (FIG. 6B). We generated a Mesp1-Luc reporter to screen with a higher throughput the molecules controlling Mesp1 expression.

1. A method for isolating multipotent cardiovascular progenitors (MCPs) from a group of stem cells comprising:
a) culturing of mammalian stem cells in a medium comprising suitable agents allowing their proliferation and maintaining their pluripotency,
b) differentiating the mammalian stem cells obtained in step a) towards cardiovascular progenitors cells, and
c) isolating those cells of step b) that express the following markers: Flk1, PDGFRa, and CXCR4,
wherein said stem cells are preferably embryonic stem cells (ES cells), preferably human embryonic stem cells, pluripotent stem cells, haematopoietic stem cells, totipotent stem cells, mesenchymal stem cells, induced pluripotent stem cells (iPS) or adult stem cells, adult heart, epicardial, vessel or muscular cells.

2. The method according to claim 1, wherein the isolation step is performed by means of cell-sorting using labeled binding molecules, such as fluorescently labeled, magnetically labeled or density labeled binding molecules, such as binding molecules selected from the group consisting of: specific antibodies, aptamers, small molecules, peptides, carbohydrates, nucleic acids, peptide-nucleic acids, and small organic molecules.

3. The method according to claim 1, wherein said selection is done at day 3 of stem cells differentiation.

4. The method according to claim 1, wherein said isolated MCPs are capable of differentiating into both primary and secondary heart field cells.

5. A kit for isolating, visualising or identifying MCPs at day 3 of stem cell differentiation, wherein said MCPs are capable of differentiating into both primary and secondary heart field cells, comprising:
a) binding molecule(s) specific for the Flk1 marker on the cell surface of a cell,
b) binding molecule(s) specific for the PDGFRa marker on the cell surface of a cell, and
c) binding molecule(s) specific for the CXCR4 marker on the cell surface of a cell.

6. The kit according to claim 5, wherein the binding molecules are selected from the group consisting of specific antibodies, aptamers, small molecules, peptides, carbohydrates, nucleic acids, peptide-nucleic acids, and small organic molecules.

7. The kit according to claim 5, wherein the binding molecules are detectably labeled, preferably fluorescently labeled, magnetically labeled or density labeled.

8. A substantially purified population of MCPs obtained by the method according to claim 1, expressing the following markers on their cell surface: Flk1, PDGFRa, and CXCR4, and capable of differentiating into both primary and secondary heart field cells.

9. A composition comprising the substantially pure population of human cardiovascular precursor cells according to claim 8.

10. A method of generating cardiovascular cells such as cardiomyocytes, endothelial cells, and vascular smooth muscle cells comprising the steps of:
a) culturing MCPs obtained according to claim 1, and
b) allowing said MCP cells to differentiate due to the endogenous expression of Mesp-1.

11. A composition comprising a population of cardiovascular cells produced by the method of claim 10.

12. A method of cardiovascular cell replacement comprising administering to a subject in need of such replacement a composition comprising a population of MCPs expressing the following markers on their cell surface: Flk1, PDGFRa, and CXCR4, and capable of differentiating into both primary and secondary heart field cells, or a composition according to claim 11.

13. A method of treating a disorder characterized by insufficient cardiac function comprising administering to a subject in need of such treatment a composition comprising a population of MCPs expressing the following markers on their cell surface: Flk1, PDGFRa, and CXCR4, and capable of differentiating into both primary and secondary heart field cells or cardiovascular cells obtained according to the method of claim 10.

14. A method for performing cellular therapy, comprising the steps of: a) providing MCPs expressing the following markers on their cell surface: Flk1, PDGFRa, and CXCR4, and capable of differentiating into both primary and secondary heart field cells, or cardiovascular cells obtained according to the method of claim 10, and b) injecting said cells into the heart or the vasculature of the subject in need thereof allowing exogenous or autologous cell therapy.

15. The method according to claim 14, wherein said cardiovascular function is preferably disturbed due to a disease or disorder selected from the group consisting of: Congenital Heart Disease, such as malformations and misplacements of cardiac structures, acquired heart and vascular diseases, such as myocardial infarction, cardiac hypertrophy and cardiac arrhythmia and cardiovascular damage due to trauma.

16. A method for identifying an extrinsic factor that promotes MCP-differentiation comprising the steps of:
a) allowing cells to differentiate into MCPs according to the method of claim 1, in the presence or absence of said extrinsic factor, and
b) analysing the effect of the extrinsic factor on MCP-differentiation by comparing the number of MCP cells formed in the presence and absence of said extrinsic factor, based on the expression of markers Flk1, PDGFRa, and CXCR4 in said cells.

17. An assay for determining the pharmacological properties and/or the toxicity of a chemical compound or pharmacological agent based on the production of cells obtained by the method of claim 1, comprising the steps of:
a) allowing cells to differentiate into MCPs according to the method of claim 1, in the presence or absence of said chemical compound or pharmacological agent, and
b) analysing the effect of the chemical compound or pharmacological agent on MCP-differentiation by comparing the number of MCP cells formed in the presence and absence of said extrinsic factor, based on the expression of markers Flk1, PDGFRa, and CXCR4 in said cells.

18. A method for specifying and/or differentiating Mesp1 expressing MCPs into a particular subset of cardiovascular lineages such as cardiomyocytes, vascular or endothelial cells, by inducing the expression of Flk1, PDGFRa and CXCR4 at day 3 of ES cell differentiation.

19. (canceled)

20. A method of targeting endogenous cardiovascular progenitors in a subject in need thereof, comprising the step of modulating the expression of Flk1, PDGFRa and CXCR4 in said cells in order to restore cardiac function.

21. The method according to claim 20, wherein said cardiovascular function is disturbed due a disease or disorder selected from the group consisting of: Congenital Heart Disease, such as malformations and misplacements of cardiac structures, acquired heart and vascular diseases, such as myocardial infarction, cardiac hypertrophy and cardiac arrhythmia and cardiovascular damage due to trauma.
